(19) United States
(12) Patent Application Publication
(10) Pub. No.: US 2025/0206719 A1
(43) Pub. Date: Jun. 26, 2025

(54) ALBICIDIN DERIVATIVES, THEIR USE AND SYNTHESIS

(71) Applicant: TECHNISCHE UNIVERSITÄT BERLIN, Berlin (DE)

(72) Inventors: Roderich SÜSSMUTH, Berlin (DE); John WESTON, Kelkheim (DE); Kay HOMMERNICK, Berlin (DE); Marcel KULIKE-KOCZULA, Berlin (DE); Lieby ZBOROVSKY, Berlin (DE); Leonardo KLEEBAUER, Berlin (DE)

(73) Assignee: TECHNISCHE UNIVERSITÄT BERLIN, Berlin (DE)

(21) Appl. No.: 18/850,028

(22) PCT Filed: Mar. 23, 2023
(86) PCT No.: PCT/EP2023/057514

§ 371 Date: Sep. 23, 2024

(30) Foreign Application Priority Data
Mar. 23, 2022 (EP) 22163715.0

(51) Int. Cl. C07D 401/14; A61K 31/4439; A61K 31/444; A61K 31/4709; A61K 31/4725; A61K 31/4741; A61K 31/498; A61P 31/04; C07D 401/12; C07D 405/14; C07D 417/14; C07D 471/04; C07D 491/056

(52) U.S. Cl. CPC C07D 401/14; A61K 31/4439; A61K 31/444; A61K 31/4709; A61K 31/4725; A61K 31/4741; A61K 31/498; A61P 31/04; C07D 401/12; C07D 405/14; C07D 417/14; C07D 471/04; C07D 491/056

(57) ABSTRACT

Please cancel the abstract of this application and replace it with the following amended abstract presented in clean form according to the procedures outlines in MPEP 714(II)(B):

It is provided a chemical compound according to general formula (1)

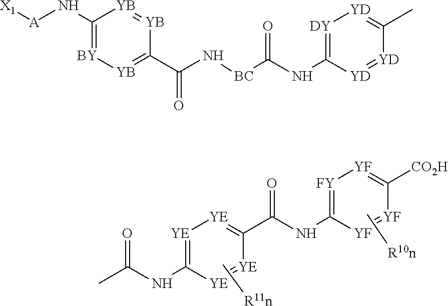

### CROSS-REFERENCE TO A RELATED APPLICATION

This application is the United States national phase of International Patent Application No. PCT/EP2023/057514, filed on Mar. 23, 2023, an claims priority of European Patent Application No. 22 163 715.0, filed on Mar. 23, 2022, the disclosure of which are hereby incorporated by reference in their entireties.

The disclosure relates to albicidin derivatives.

### BACKGROUND

Albicidin is a natural product, isolated from Xanthomonas albilineans and heterologously expressed in Xanthomonas axonopodis pv vesicatoria, Its structure (see below) is based on peptides and amino acids, but it does not contain any proteinogenic amino acids.

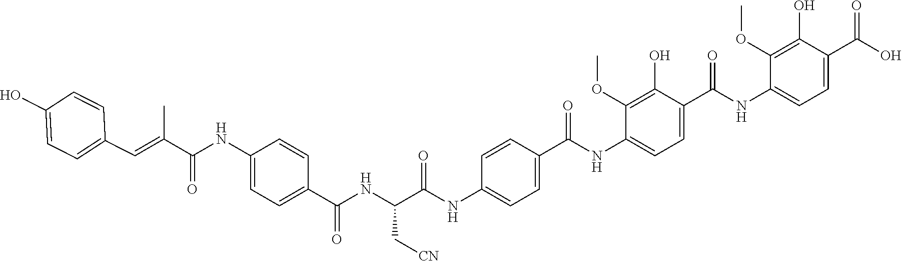 [SMILES: COc1c(NC(=O)c2ccc(NC(=O)c3ccc(NC(=O)[C@H](CC#N)NC(=O)c4ccc(NC(=O)/C(C)=C/c5ccc(O)cc5)cc4)cc3)c(OC)c2O)ccc(C(=O)O)c1O]

Albicidin is, on the one hand, a causative agent of the leaf scald disease in sugar cane and on the other hand a DNA-gyrase-inhibitor of prokaryotic cells (gram-positive and -negative). The mentioned properties make the natural product albicidin a potential antibiotic.

The known molecular structure of albicidin and available synthetic routes allows the development of a plurality of novel derivatives that may exhibit potential antimicrobial activities.

Since the exact interaction of albicidin with gyrase is not yet fully understood, structure-activity studies are of great importance to find out which structural elements influence the inhibition of gyrase. The aim is to develop a structure that, on the one hand, inhibits as wide a range of bacteria as possible, especially those of the ESKAPE group. On the other hand, the structure should overcome existing resistances.

Another important point is the pharmacological profile, which must be adapted so that the active substance reaches its site of action in the body. For this purpose, the active substance must ensure sufficient bioavailability, duration of action, metabolic stability, selectivity and good tolerability as well as low toxicity. These properties are determined by the so-called ADME parameters (absorption, distribution, metabolism and excretion), which in turn determine the pharmacokinetics of the substance. Decisive for optimal pharmacokinetics are physicochemical properties of the active substance such as polarity, lipophilicity and solubility.

But molecular size and flexibility also play a role, two parameters that can indirectly influence the physicochemical properties. To prevent the failure of an active ingredient at a late stage of development, it is important to integrate pharmacokinetics already in the lead structure optimization. The aim is to adjust the pharmacological profile of albicidin with regard to good oral bioavailability and optimisation of the spectrum of activity with increased bioactivity. Albicidin's poor water solubility is already evident in its structure. The calculated octanol-water partition coefficient of albicidin is clogP=5.2.

The related property of solubility is one of the most important parameters for improving the drug. It is known that bacterial efflux systems pump out amphiphilic and lipophilic molecules more effectively than hydrophilic compounds. Moreover, in many cases, high lipophilicity is also related to increased plasma protein binding (PPB), through for example human serum albumin, lipoproteins and glycoproteins, reducing the amount of active, unbound drug. However, excessive polarity may prevent albicidin from penetrating the cytoplasmic membrane.

### SUMMARY

The problem underlying the proposed solution is therefore provision of new compounds, which comprise antibiotic properties, solubility and bioavailability, method of their synthesis and their use. This problem is attained by a compound having features as described herein.

### Terms and Definitions

The term “purity” as used in the context of the present specification with respect to a preparation of a certain compound refers to the content of said compound relative to the sum of all compounds contained in the preparation. The term “compound” in this context is to be understood as a compound according to the solution (or any specific embodiments thereof) as well as any salts, hydrates or solvates thereof. Thus, the respective salts, hydrates or solvates are not considered as impurities according to the previous definition. The “purity” of a compound may be determined using elemental analysis, HPLC analysis using UV diode array detection also in combination with mass spectrometry detection, or quantitative NMR analysis.

The term “substituted” refers to the addition of a substituent group to a parent moiety. “Substituent groups” can be protected or unprotected and can be added to one available site or to many available sites in a parent moiety.

As used herein the term “aryl” refers to a hydrocarbon with alternating double and single bonds between the carbon atoms forming an aromatic ring structure, in particular a ten (C₁₀) membered ring. The term “heteroaryl” refers to aromatic structures comprising a ten membered ring or polyring structure, comparable to aryl compounds, in which at least one member is an oxygen or a nitrogen or a sulphur atom.

### Description of the Solution

According to a first aspect, the solution relates to compounds having a molecular structure as defined by formula (1)

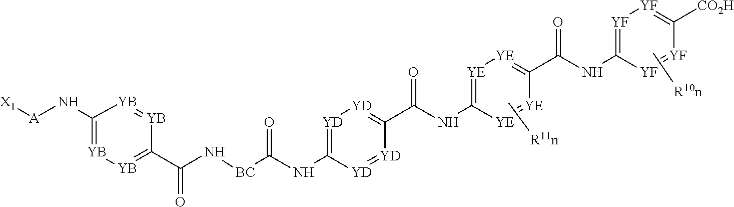

With A being CH₂ or CO
with X₁ being
   a substituted or unsubstituted 9-10 membered bicyclic system with both rings aromatic, or, one ring being aromatic and the other ring containing at least one double bond or one ring being aromatic and the other ring being alicyclic,
   a substituted or unsubstituted a 9-10 membered bicyclic heterocyclic system with both rings aromatic or one ring being aromatic and the other ring containing at least one double bond or one ring being aromatic and the other ring being alicyclic, wherein at least one heteroatom is N, S or O,
b) with BC being

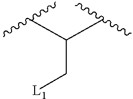

with L₁ being a substituted or unsubstituted C₅-C₆ aromatic heterocycle,
c) with n of R¹⁰ₙ and n of R¹¹ₙ being independently from each other 0, 1, 2, 3 or 4, in particular n of R¹⁰ₙ and n of R¹¹ₙ being 0, 1, 2 or 3, more particular n of R¹⁰ₙ and n of R¹¹ₙ being 1 and 2;
   with each R¹⁰ and R¹¹ being selected independently from any other R¹⁰ and R¹¹ from —OH, —F, —Cl, —Br, —I, —CCH, —CN, —OC₁-C₆ alkyl, in particular from —OH, —F, —OCH₃, —OC₂H₅, -OiC₃H₇, -OnC₃H₇, —OCF₃; and
d) with YB, YD, YE and YF being independently from each other N, CF, CCl or CH, in particular N and CH.

In a preferred embodiment, A is CO:

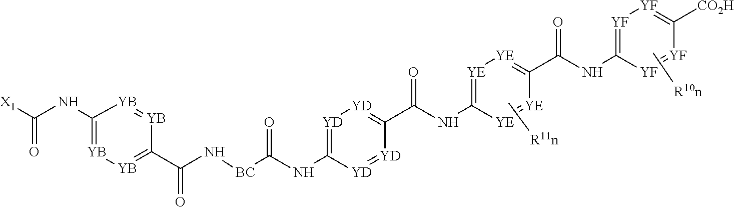

It is to be understood that if X1 is
a substituted or unsubstituted 9-10 membered bicyclic system with one ring being aromatic and the other ring containing at least one double bond or one ring being aromatic and the other ring being alicyclic, or
a substituted or unsubstituted a 9-10 membered bicyclic heterocyclic system with one ring being aromatic and the other ring containing at least one double bond or one ring being aromatic and the other ring being alicyclic, wherein at least one heteroatom is N, S or O,
   the aromatic ring of the 9-10 membered bicyclic system is not adjacent to the amide bond; i.e. has no direct bond to the rest of the albicidin derivative. In other words, the linkage between the bicyclic system and the peptide bond occurs solely on the alicyclic ring or the ring containing at least one double bond of the bicyclic system. This applies in particular to the case wherein X1 is a 10 membered bicyclic system.

It is further to be understood that X1 is not

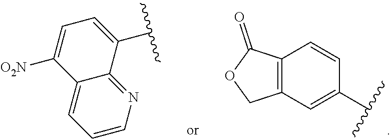 [SMILES: *c1ccc([N+](=O)[O-])c2cccnc12 ; *c1ccc2c(c1)COC2=O]

These moieties are thus exempted. In particular, the above nitro quinoline, where the first 6-membered carbocycle adjacent to the peptide bond is connected to the terminal 6-membered heterocycle in ortho- and meta-position to the peptide bond, is exempted. In other words, the bridgehead carbons connecting the fused bicycle of the above nitro quinoline are located in ortho- and meta-position to the peptide bond linking the bicycle to the rest of the molecule. The described connectivity of above nitro quinoline causes a non-linear structure of the N-terminal part of the molecule and the above nitro quinoline is therefore exempted.

It is to be noted that the present albicidin derivatives have a linear structure at the N-terminal part of the molecule. This applies in particular to the connectivity of a bicycle X1 where the first 6-membered cycle adjacent to A is connected to the terminal 6- or 5-membered cycle in para and meta position to the bond linking X1 to the rest of the albicidin structure.

In one embodiment, moiety X1 is a derivative of naphthalene, indene, quinoline, quinolone, oxochromene or benzazoles, benzofuranes, in particular quinoline, quinolone or benzazoles, isoquinoline, quinoxaline or quinazoline.

In one embodiment, moiety X₁ is one of the following:

### Naphthalenes and Indenes

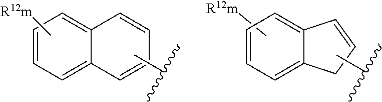

### Indoles and Isoindoles

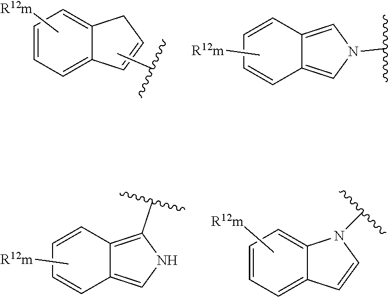

### Benzazoles

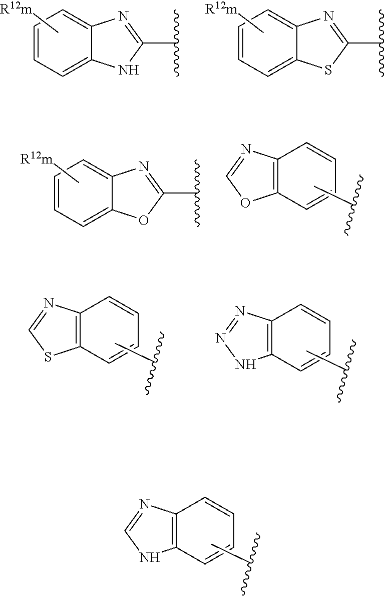

### Benzofuranes

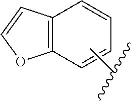

### Quinolines, Isoquinolines, Quinoxalines and Quinazolines and Related Systems

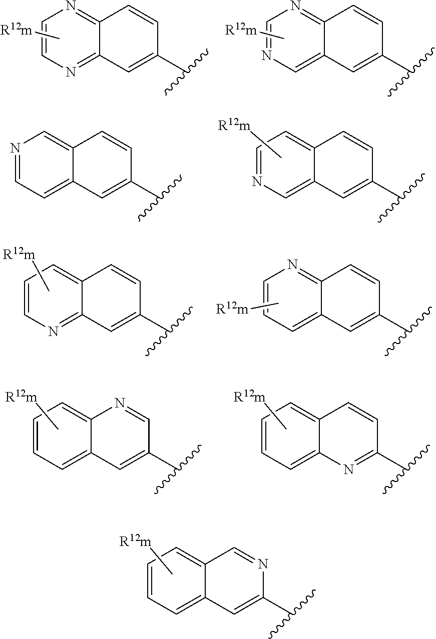

### Qinolones

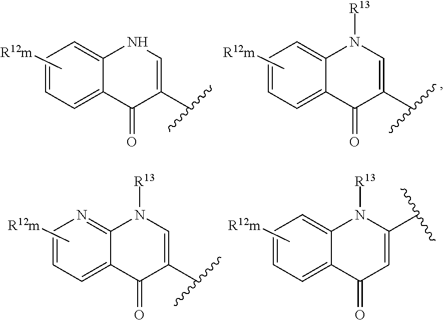

### Oxochromenes

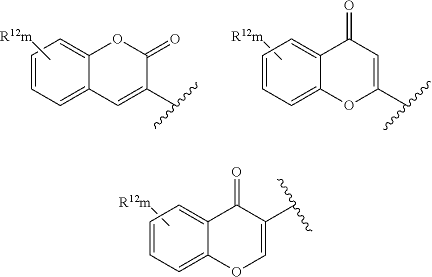

### Tetrahydroisoquinoline

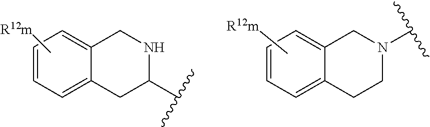

In another embodiment, moiety X1 is one of the following

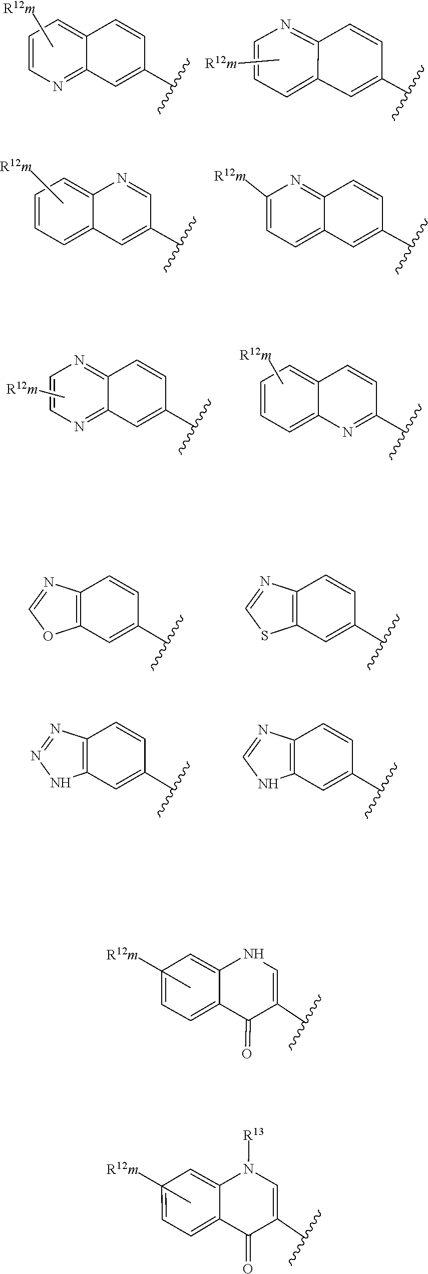

In one aspect, m of R¹² is any of 0-6, preferably 0, 1, 2, 3, 4, more preferably 0, 1, 2, 3; even more preferably 0, 1; and R¹² is selected from —OH, —OC₁-C₆ alkyl, —C₁-C₆ alkyl, —F, —Cl, —NR₂, ——(CH₂)ₙNR₂, —O(CH₂)ₙNR₂ with R being H or —C₁-C₆ alkyl and n being 1 or 2, in particular from OH, —CH₃, —C₂H—₅, —OCH₃, —OC₂H—₅, or two of R¹² form an acetal moiety. In an embodiment R¹² is selected from —OH, —CH₃, —C₂H₅, —OCH₃, —OC₂H₅, in particular—OH and —CH₃, or two of R¹² form—O—CH₂—O— moiety.

In a further aspect R¹³ is selected from —C₁-C₆ alkyl, in particular—CH₃, —C₂H₅, —C₃H₇.

In a preferred embodiment, moiety X1 is one of the following

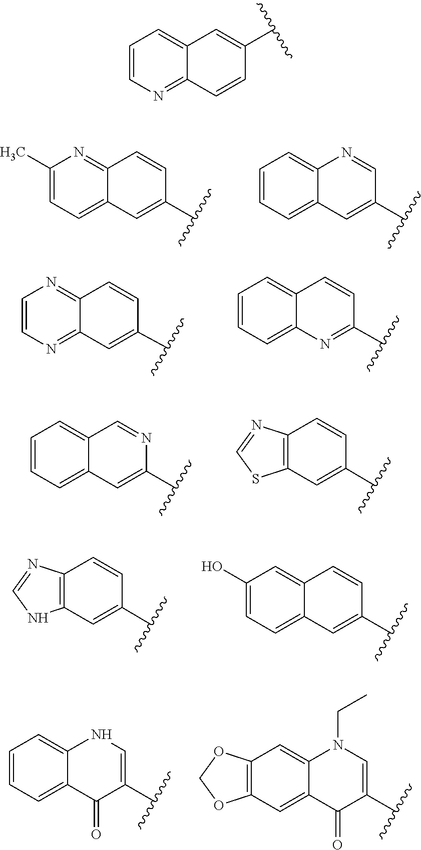

In a most preferred embodiment, moiety X1 is one of the following:

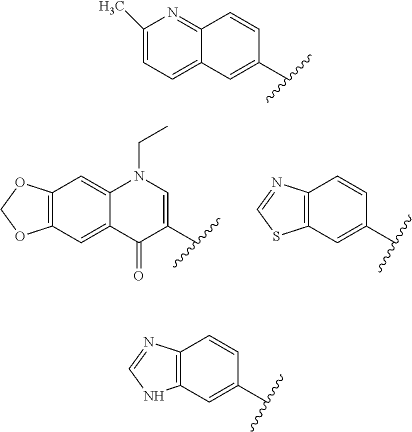 [SMILES: *c1ccc2nc(C)ccc2c1 ; *c1cn(CC)c2cc3c(cc2c1=O)OCO3 ; *c1ccc2ncsc2c1 ; *c1ccc2nc[nH]c2c1]

It is to be noted that in one case a naphthyl with at least one OH substituent and an indole with at least one OH-substituent may be disclaimed as moiety X1.

In one embodiment, the moiety L₁ is a five membered aromatic N-heterocycle. In a preferred embodiment, moiety L1 is an imidazole or triazole, most preferably an unsubstituted triazole. In another preferred embodiment, n of R¹⁰ₙ and n of R¹¹ₙ being 0, 1, 2, 3 or 4, in particular n of R¹⁰ₙ and n of R¹¹ₙ being 0, 1, 2 or 3, and with each R¹⁰ and with each R¹¹ independently from any other R¹⁰ being selected from —OH, —OCH₃, —OC₂H₅ or -OiPr, particularly with one R¹⁰ or R¹¹ being —OH and the other R¹⁰ or R¹¹ being —OCH₃, —OC₂H₅ or -OiPr respectively. In one specific embodiment R10 is H and R11 is one of from —OH, —OCH₃, —OC₂H₅ or -OiPr.

In a preferred embodiment the present compound may be of the general formulae (1a)

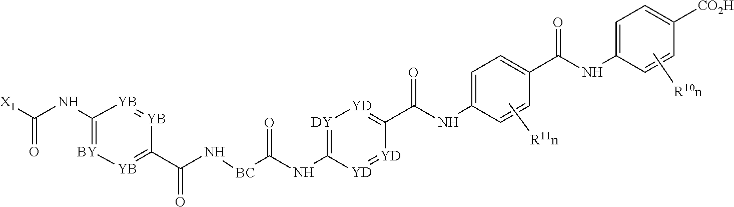

With YB being independently from each other CF, CCl or CH,
with YD being independently from each other N, CF, CCl or CH, in particular N and CH,
with X1, BC, R¹⁰ₙ and R¹¹ₙ as described previously above.

In a preferred embodiment the present compound may be of the general formulae (2)

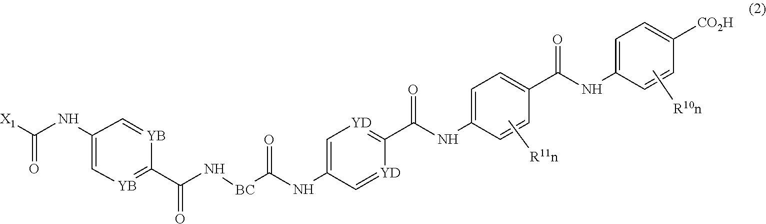

with YB being independently from each other CF, CCl or CH,
with YD being independently from each other N, CF, CCl or CH, in particular N and CH,
with X1, BC, R¹⁰ₙ and R¹¹ₙ as described previously above.

In a further preferred embodiment, the present compound may be of the general formulae (3)

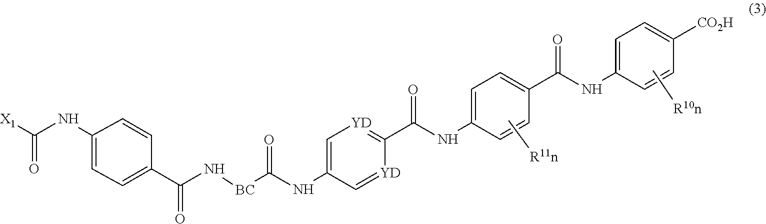

with YD being independently from each other N, CF, CCl or CH, in particular N and CH,
with X1, BC, R¹⁰ₙ and R¹¹ₙ as described previously above.

In yet another preferred embodiment, the present compound may be of the general formulae (4)

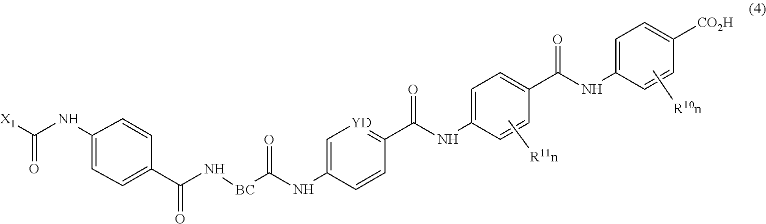

with YD being N,
with L1 being a triazole or imidazole, preferably triazole,
with X1, Ron and R¹¹ₙ as described previously above.

In an even more preferred embodiment, the present compound may be of general formulae (5)

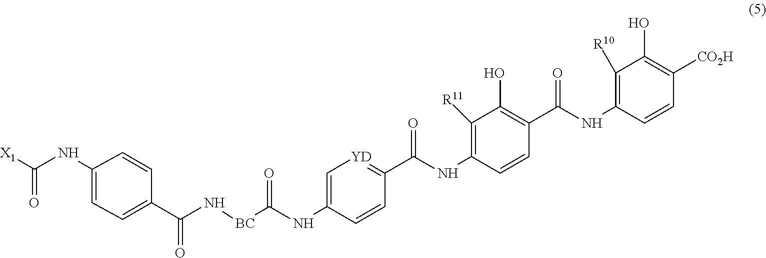

with YD being N,
with L1 being a triazole or imidazole, preferably triazole,
with X1, Ron and R¹¹ₙ as described previously above.

In a still more preferred embodiment, the present compound may be of general formulae (6)

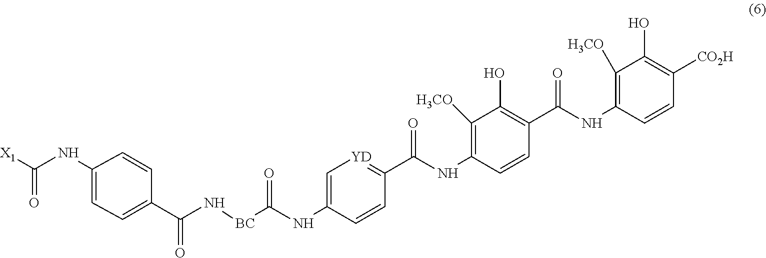

with YD being N,
with L1 being a triazole or imidazole, preferably triazole,
with X1 as described previously above.

It is further to be understood that the present compounds have at least one of the following properties:
MIC values against gram negative bacteria, such as E. coli or the genus Salmonella, such as S. typhimurium, between 0.01 and 0.15, preferably between 0.015 and 0.125, more preferably between 0.015 and 0.07;
MIC values against gram positive bacteria, in particular of the genus Bacillus, such as Bacillus subtilis, and of the genus Micrococcus, such as Micrococcus luteus, between 0.01 and 0.15, preferably between 0.015 and 0.125, more preferably between 0.015 and 0.07;
MIC values against bacteria of the genus Mycobacterium, such as Mycobacterium phlei, between 0.2 and 8, preferably between 0.5 and 4, more preferably between 0.5 and 2;
MIC values against gram negative bacteria, such as E. coli, in the presence of 50% human serum between 0.2 and 8, preferably between 0.5 and 4, more preferably between 0.5 and 2, even more preferably between 0.5 and 1.0.

Particular embodiments of the solution are one of the following compounds:

### Compound 1:

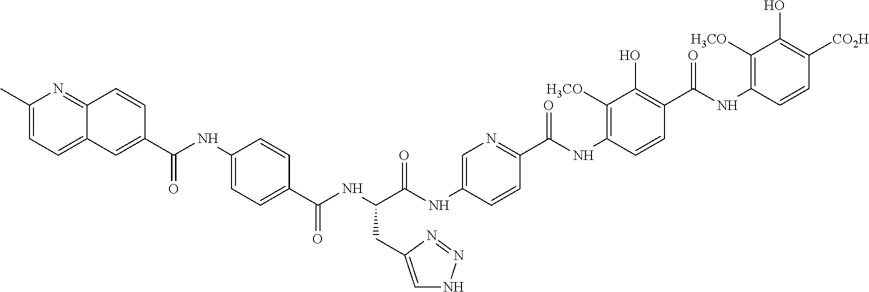 [SMILES: COc1c(NC(=O)c2ccc(NC(=O)c3ccc(NC(=O)[C@H](Cc4c[nH]nn4)NC(=O)c4ccc(NC(=O)c5ccc6nc(C)ccc6c5)cc4)cn3)c(OC)c2O)ccc(C(=O)O)c1O]

### Compound 2:

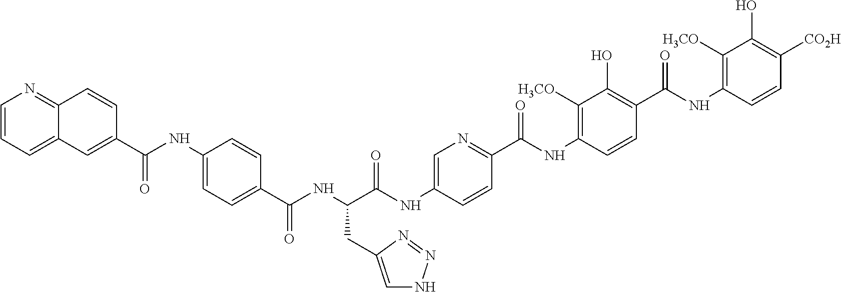 [SMILES: COc1c(NC(=O)c2ccc(NC(=O)c3ccc(NC(=O)[C@H](Cc4c[nH]nn4)NC(=O)c4ccc(NC(=O)c5ccc6ncccc6c5)cc4)cn3)c(OC)c2O)ccc(C(=O)O)c1O]

### Compound 3:

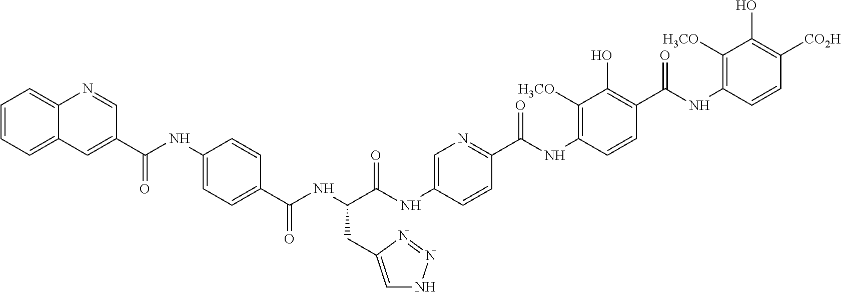 [SMILES: COc1c(NC(=O)c2ccc(NC(=O)c3ccc(NC(=O)[C@H](Cc4c[nH]nn4)NC(=O)c4ccc(NC(=O)c5cnc6ccccc6c5)cc4)cn3)c(OC)c2O)ccc(C(=O)O)c1O]

### Compound 4:

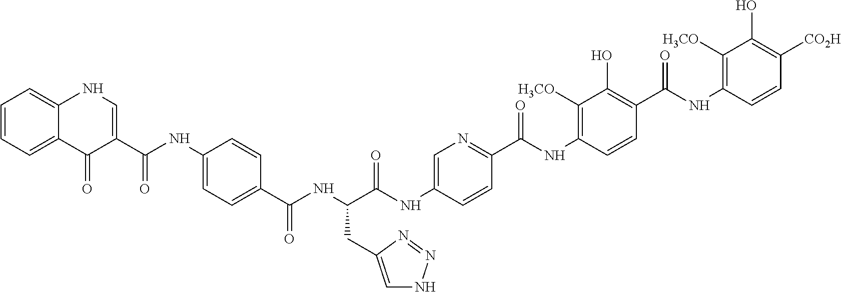 [SMILES: COc1c(NC(=O)c2ccc(NC(=O)c3ccc(NC(=O)[C@H](Cc4c[nH]nn4)NC(=O)c4ccc(NC(=O)c5c[nH]c6ccccc6c5=O)cc4)cn3)c(OC)c2O)ccc(C(=O)O)c1O]

### Compound 5:

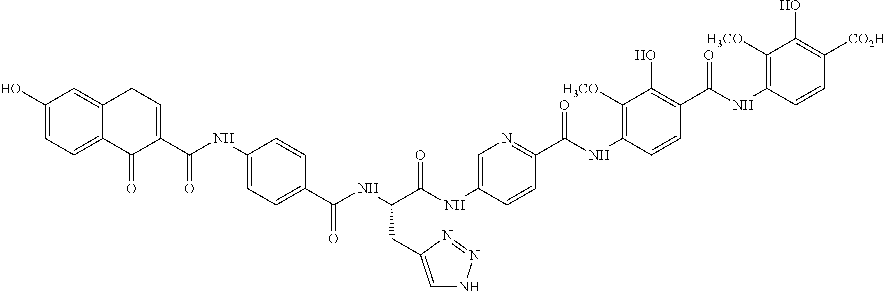 [SMILES: COc1c(NC(=O)c2ccc(NC(=O)c3ccc(NC(=O)[C@H](Cc4c[nH]nn4)NC(=O)c4ccc(NC(=O)C5=CCc6cc(O)ccc6C5=O)cc4)cn3)c(OC)c2O)ccc(C(=O)O)c1O]

### Compound 6:

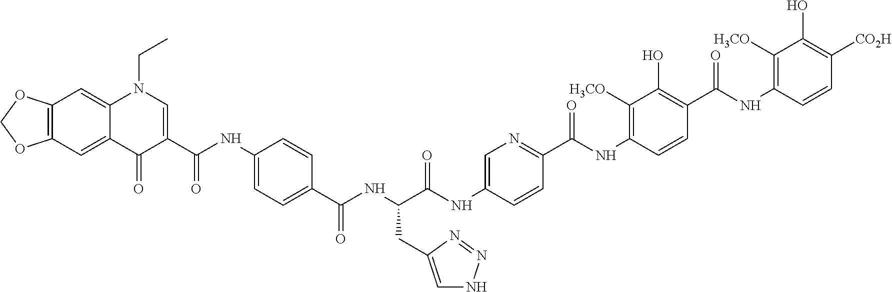 [SMILES: CCn1cc(C(=O)Nc2ccc(C(=O)N[C@@H](Cc3c[nH]nn3)C(=O)Nc3ccc(C(=O)Nc4ccc(C(=O)Nc5ccc(C(=O)O)c(O)c5OC)c(O)c4OC)nc3)cc2)c(=O)c2cc3c(cc21)OCO3]

### Compound 7:

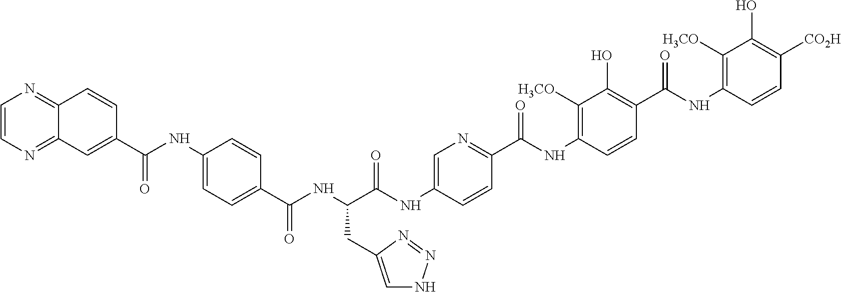 [SMILES: COc1c(NC(=O)c2ccc(NC(=O)c3ccc(NC(=O)[C@H](Cc4c[nH]nn4)NC(=O)c4ccc(NC(=O)c5ccc6nccnc6c5)cc4)cn3)c(OC)c2O)ccc(C(=O)O)c1O]

### Compound 8:

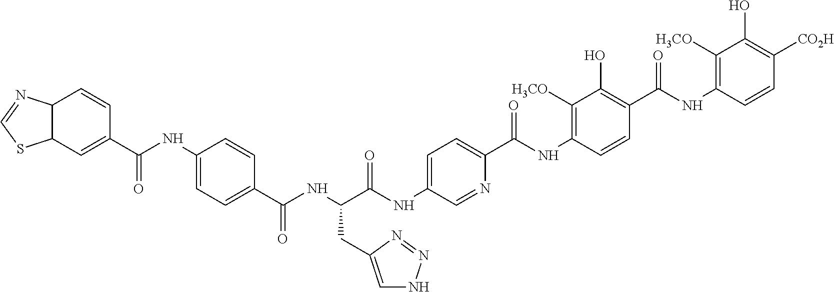 [SMILES: COc1c(NC(=O)c2ccc(NC(=O)c3ccc(NC(=O)[C@H](Cc4c[nH]nn4)NC(=O)c4ccc(NC(=O)C5=CC6SC=NC6C=C5)cc4)cn3)c(OC)c2O)ccc(C(=O)O)c1O]

### Compound 9:

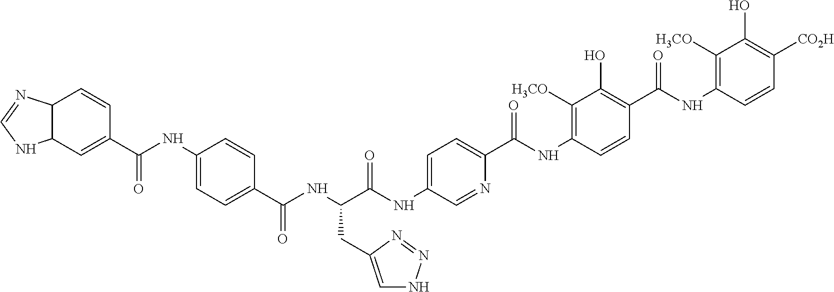 [SMILES: COc1c(NC(=O)c2ccc(NC(=O)c3ccc(NC(=O)[C@H](Cc4c[nH]nn4)NC(=O)c4ccc(NC(=O)C5=CC6NC=NC6C=C5)cc4)cn3)c(OC)c2O)ccc(C(=O)O)c1O]

### Compound 10:

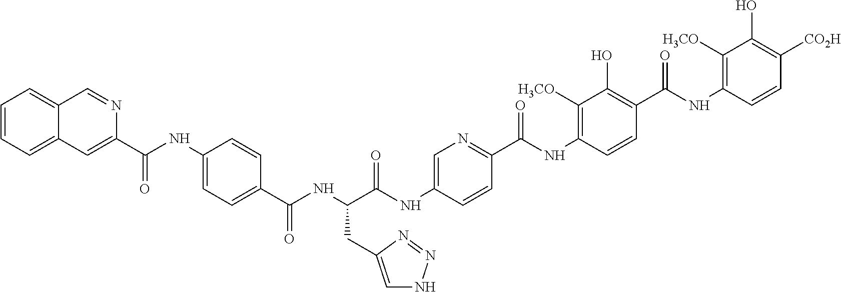 [SMILES: COc1c(NC(=O)c2ccc(NC(=O)c3ccc(NC(=O)[C@H](Cc4c[nH]nn4)NC(=O)c4ccc(NC(=O)c5cc6ccccc6cn5)cc4)cn3)c(OC)c2O)ccc(C(=O)O)c1O]

### Compound 11:

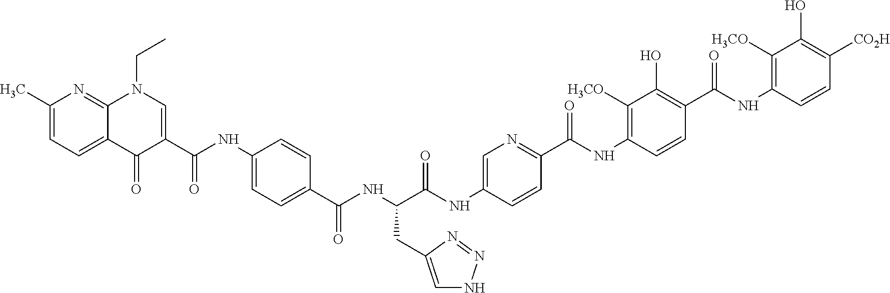 [SMILES: CCn1cc(C(=O)Nc2ccc(C(=O)N[C@@H](Cc3c[nH]nn3)C(=O)Nc3ccc(C(=O)Nc4ccc(C(=O)Nc5ccc(C(=O)O)c(O)c5OC)c(O)c4OC)nc3)cc2)c(=O)c2ccc(C)nc21]

### Compound 12:

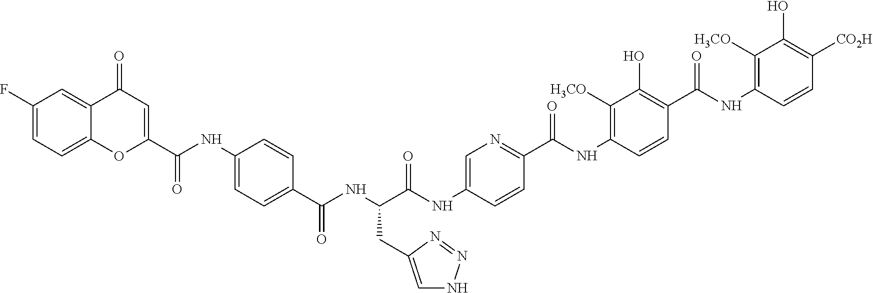 [SMILES: COc1c(NC(=O)c2ccc(NC(=O)c3ccc(NC(=O)[C@H](Cc4c[nH]nn4)NC(=O)c4ccc(NC(=O)c5cc(=O)c6cc(F)ccc6o5)cc4)cn3)c(OC)c2O)ccc(C(=O)O)c1O]

### Compound 13:

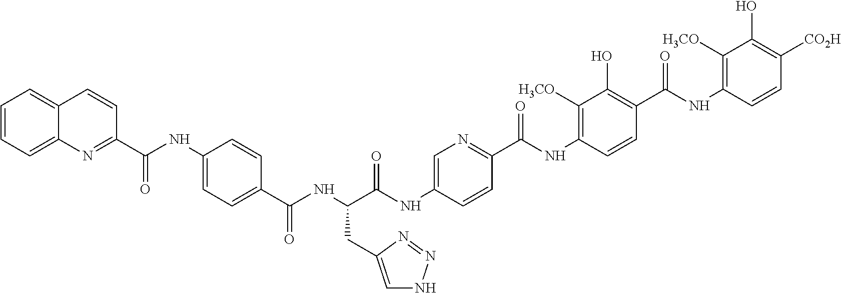 [SMILES: COc1c(NC(=O)c2ccc(NC(=O)c3ccc(NC(=O)[C@H](Cc4c[nH]nn4)NC(=O)c4ccc(NC(=O)c5ccc6ccccc6n5)cc4)cn3)c(OC)c2O)ccc(C(=O)O)c1O]

### Compound 14:

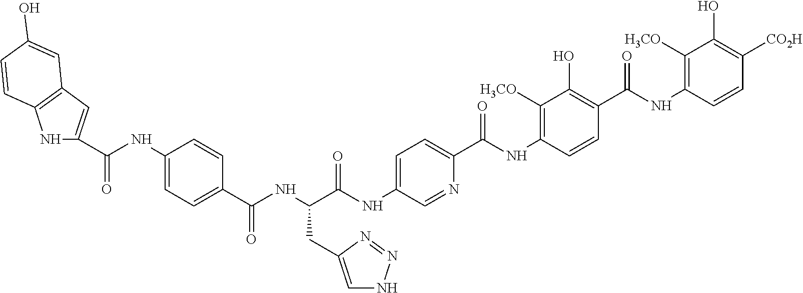 [SMILES: COc1c(NC(=O)c2ccc(NC(=O)c3ccc(NC(=O)[C@H](Cc4c[nH]nn4)NC(=O)c4ccc(NC(=O)c5cc6cc(O)ccc6[nH]5)cc4)cn3)c(OC)c2O)ccc(C(=O)O)c1O]

### Compound 15:

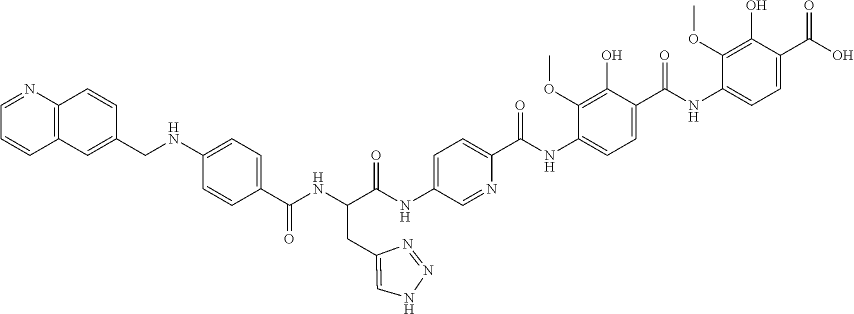 [SMILES: COc1c(NC(=O)c2ccc(NC(=O)c3ccc(NC(=O)C(Cc4c[nH]nn4)NC(=O)c4ccc(NCc5ccc6ncccc6c5)cc4)cn3)c(OC)c2O)ccc(C(=O)O)c1O]

### Compound 16

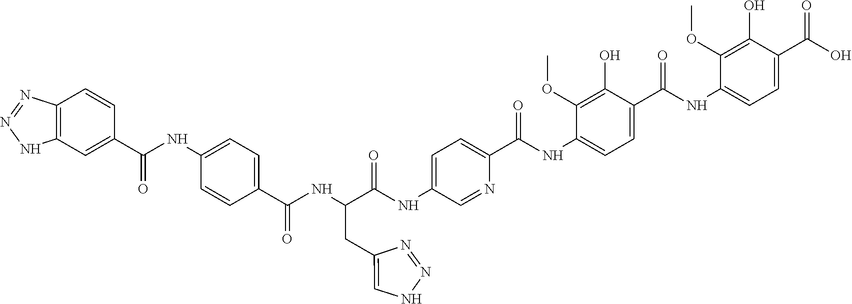 [SMILES: COc1c(NC(=O)c2ccc(NC(=O)c3ccc(NC(=O)C(Cc4c[nH]nn4)NC(=O)c4ccc(NC(=O)c5ccc6nn[nH]c6c5)cc4)cn3)c(OC)c2O)ccc(C(=O)O)c1O]

The compounds of the proposed solution may be used in a method of treatment of diseases, in particular for use in a method of treatment of bacterial infections caused by gram-negative or gram-positive bacterial strains.

The bacterial infection may be an infection (by a gram-negative bacterium) caused by one of the genus Acinetobacter, Bordatella, Borellia, Brucella, Camphylobacter, Chlamydia, Chlamydophila, Enterobacter, Escherichia, Francisella, Haemophilus, Helicobacter, Klebsiella, Legionella, Leptospira, Morganella Moraxella, Neisseria, Proteus, Pseudomonas, Rickettsia, Shigella, Salmonella, Stenotrophomonas, Treponema or Yersinia, in particular an infection caused by one of the genus Escherichia, Enterobacter, Salmonella, Klebsiella, Pseudomonas, Haemophilus, Shigella, Proteus or Morganella.

In a further embodiment the bacterial infection is an infection caused
by a gram-positive bacterium, particularly an infection by one of the genus Bacillus, Clostridium, Corynebacterium, Enterococcus, Listeria, Micrococcus, Staphylococcus or Streptococcus, further in particular by one of the genus of Staphylococcus, Streptococcus, Bacillus or Micrococcus or
by a bacterium of the family of Mycobacteriaceae, in particular of the genus Mycobacterium, further in particular an infection by one of Mycobacterium tuberculosis, Mycobacterium leprae, Mycobacterium ulcerans or Mycobacterium avium, or
by a bacterium of the family of Mycoplasmataceae, in particular of the genus Mycoplasma, further in particular an infection by Mycoplasma pneumonia.

For this purpose, the present compounds may be provided in a pharmaceutical acceptable form. Pharmaceutically acceptable salts of the present compounds mean both their organic and inorganic salts as described in Remington's Pharmaceutical Sciences (17^{th} edition, page 1418 (1985)). Because of the physical and chemical stability and the solubility, preference is given for acidic groups inter alia to sodium, potassium, calcium and ammonium salts; preference is given for basic groups inter alia to salts of maleic acid, fumaric acid, succinic acid, malic acid, tartaric acid, methylsulfonic acid, hydrochloric acid, sulfuric acid, phosphoric acid or of carboxylic acids or sulfonic acids, for example as hydrochlorides, hydrobromides, phosphates, sulfates, methanesulfonates, acetates, lactates, maleates, fumarates, malates, gluconates, and salts of amino acids, of natural bases or carboxylic acids. The preparation of pharmaceutically acceptable salts from compounds of the formula (I) which are capable of salt formation, including their stereoisomeric forms, takes place in a manner known per se. The present compounds form stable alkali metal, alkaline earth metal or optionally substituted ammonium salts with basic reagents such as hydroxides, carbonates, bicarbonates, alcoholates and ammonia or organic bases, for example trimethyl- or triethylamine, ethanolamine, diethanolamine or triethanolamine, trometamol or else basic amino acids, for example lysine, ornithine or arginine. Where the compounds of the formula (I) have basic groups, stable acid addition salts can also be prepared with strong acids. Suitable pharmaceutically acceptable acid addition salts of the compounds of the solution are salts of inorganic acids such as hydrochloric acid, hydrobromic, phosphoric, metaphosphoric, nitric and sulfuric acid, and of organic acids such as, for example, acetic acid, benzenesulfonic, benzoic, citric, ethanesulfonic, fumaric, gluconic, glycolic, isethionic, lactic, lactobionic, maleic, malic, methanesulfonic, succinic, p-toluenesulfonic and tartaric acid. The hydrochloride salt is a preferred salt.

In a preferred embodiment formulations of the present albicidin derivatives are provided which contain cyclodextrins for improving solubility of the otherwise poorly soluble albicidin derivatives. Cyclodextrins are used in a concentration of 20-40%, preferably 25-35%, more preferably 28-30%.

Salts with a pharmaceutically unacceptable anion such as, for example, trifluoroacetate likewise belong within the framework of the solution as useful intermediates for the preparation or purification of pharmaceutically acceptable salts and/or for use in non-therapeutic, for example in vitro, applications.

The proposed solution furthermore relates to pharmaceutical preparations (or pharmaceutical compositions) which contain an effective amount of at least one of the present compounds and/or its pharmaceutically acceptable salts and a pharmaceutically acceptable carrier, i. e. one or more pharmaceutically acceptable carrier substances (or vehicles) and/or additives (or excipients). The pharmaceuticals can be administered orally, for example in the form of pills, tablets, lacquered tablets, coated tablets, granules, hard and soft gelatine capsules, solutions, syrups, emulsions, suspensions or aerosol mixtures. Administration, however, can also be carried out rectally, for example in the form of suppositories, or parenterally, for example intravenously, intramuscularly or subcutaneously, in the form of injection solutions or infusion solutions, microcapsules, implants or rods, or percutaneously or topically, for example in the form of ointments, solutions or tinctures, or in other ways, for example in the form of aerosols or nasal sprays.

The pharmaceutical preparations according to the solution are prepared in a manner known per se and familiar to one skilled in the art, pharmaceutically acceptable inert inorganic and/or organic carrier substances and/or additives being used in addition to the compound(s) of the formula (I) and/or its (their) pharmaceutically acceptable salts and/or its (their) prodrugs. For the production of pills, tablets, coated tablets and hard gelatine capsules it is possible to use, for example, lactose, corn starch or derivatives thereof, talc, stearic acid or its salts, etc. Carrier substances for soft gelatine capsules and suppositories are, for example, fats, waxes, semisolid and liquid polyols, natural or hardened oils, etc. Suitable carrier substances for the production of solutions, for example injection solutions, or of emulsions or syrups are, for example, water, saline, alcohols, glycerol, polyols, sucrose, invert sugar, glucose, vegetable oils, etc. Suitable carrier substances for microcapsules, implants or rods are, for example, copolymers of glycolic acid and lactic acid. The pharmaceutical preparations normally contain about 0.5 to about 90% by weight of the present compounds and/or their pharmaceutically acceptable salts and/or their prodrugs. The amount of the active ingredient of the formula (I) and/or its pharmaceutically acceptable salts and/or its prodrugs in the pharmaceutical preparations normally is from about 0.5 to about 1000 mg, preferably from about 1 to about 500 mg.

A prodrug is a precursor chemical compound of a biological active compound of the solution. Instead of administering the active compound or drug, a prodrug might be used instead to improve the absorption, distribution, metabolization and excretion. Prodrugs are often designed to improve bioavailability when a drug itself is poorly absorbed from the gastrointestinal tract. A prodrug may also be used to improve the selectively of the drug. This reduces adverse or unintended effects of a drug, especially important in treatments like chemotherapy, which can have severe unintended and undesirable side effects.

In addition to the active compound according to the solution and/or their pharmaceutically acceptable salts and to carrier substances, the pharmaceutical preparations can contain one or more additives such as, for example, fillers, disintegrants, binders, lubricants, wetting agents, stabilizers, emulsifiers, preservatives, sweeteners, colorants, flavourings, aromatizers, thickeners, diluents, buffer substances, solvents, solubilizers, agents for achieving a depot effect, salts for altering the osmotic pressure, coating agents or antioxidants. They can also contain two or more of the present compounds and/or their pharmaceutically acceptable salts. In case a pharmaceutical preparation contains two or more of the present compounds the selection of the individual compounds can aim at a specific overall pharmacological profile of the pharmaceutical preparation. For example, a highly potent compound with a shorter duration of action may be combined with a long-acting compound of lower potency. The flexibility permitted with respect to the choice of substituents in the present compounds allows a great deal of control over the biological and physico-chemical properties of the compounds and thus allows the selection of such desired compounds. Furthermore, in addition to at least one compound and/or its pharmaceutically acceptable salts, the pharmaceutical preparations can also contain one or more other therapeutically or prophylactically active ingredients. When using the present compounds the dose can vary within wide limits and, as is customary and is known to the physician, is to be suited to the individual conditions in each individual case. It depends, for example, on the specific compound employed, on the nature and severity of the disease to be treated, on the mode and the schedule of administration, or on whether an acute or chronic condition is treated or whether prophylaxis is carried out. An appropriate dosage can be established using clinical approaches well known in the medical art. In general, the daily dose for achieving the desired results in an adult weighing about 75 kg is from about 0.01 to about 100 mg/kg, preferably from about 0.1 to about 50 mg/kg, in particular from about 0.1 to about 10 mg/kg, (in each case in mg per kg of body weight). The daily dose can be divided, in particular in the case of the administration of relatively large amounts, into several, for example 2, 3 or 4, part administrations. As usual, depending on individual behaviour it may be necessary to deviate upwards or downwards from the daily dose indicated.

The compounds of the solution may also exist in various polymorphous forms, for example as amorphous and crystalline polymorphous forms. All polymorphous forms of the compounds of the solution belong within the framework of the solution and are a further aspect of the solution.

The compounds of the solution may be present as optical isomers or as mixtures thereof. The solution relates both to the pure isomers and all possible isomeric mixtures and is hereinafter understood as doing so, even if stereochemical details are not specifically mentioned in every case. Enantiomeric mixtures of compounds of the general formula 1, which are obtainable by the process or any other way, may be separated in known manner—on the basis of the physical-chemical differences of their components—into pure enantiomers, for example by fractional 19rystallization, distillation and/or chromatography, in particular by preparative HPLC using a chiral HPLC column.

According to the solution, apart from separation of corresponding isomer mixtures, generally known methods of diastereoselective or enantioselective synthesis can also be applied to obtain pure diastereoisomers or enantiomers, e.g. by carrying out the method described hereinafter and using educts with correspondingly suitable stereochemistry.

It is advantageous to isolate or synthesize the biologically more active isomer, provided that the individual compounds have different biological activities.

The solution is explained in more detail by means of the following examples.

### Methods of Synthesis

One general procedure for the synthesis of albicidin-derivatives with variations to amide bonds may comprise the steps according to the following procedure.

### Compound 1

Compound 1 is synthesized in a multistep synthesis route as follows:

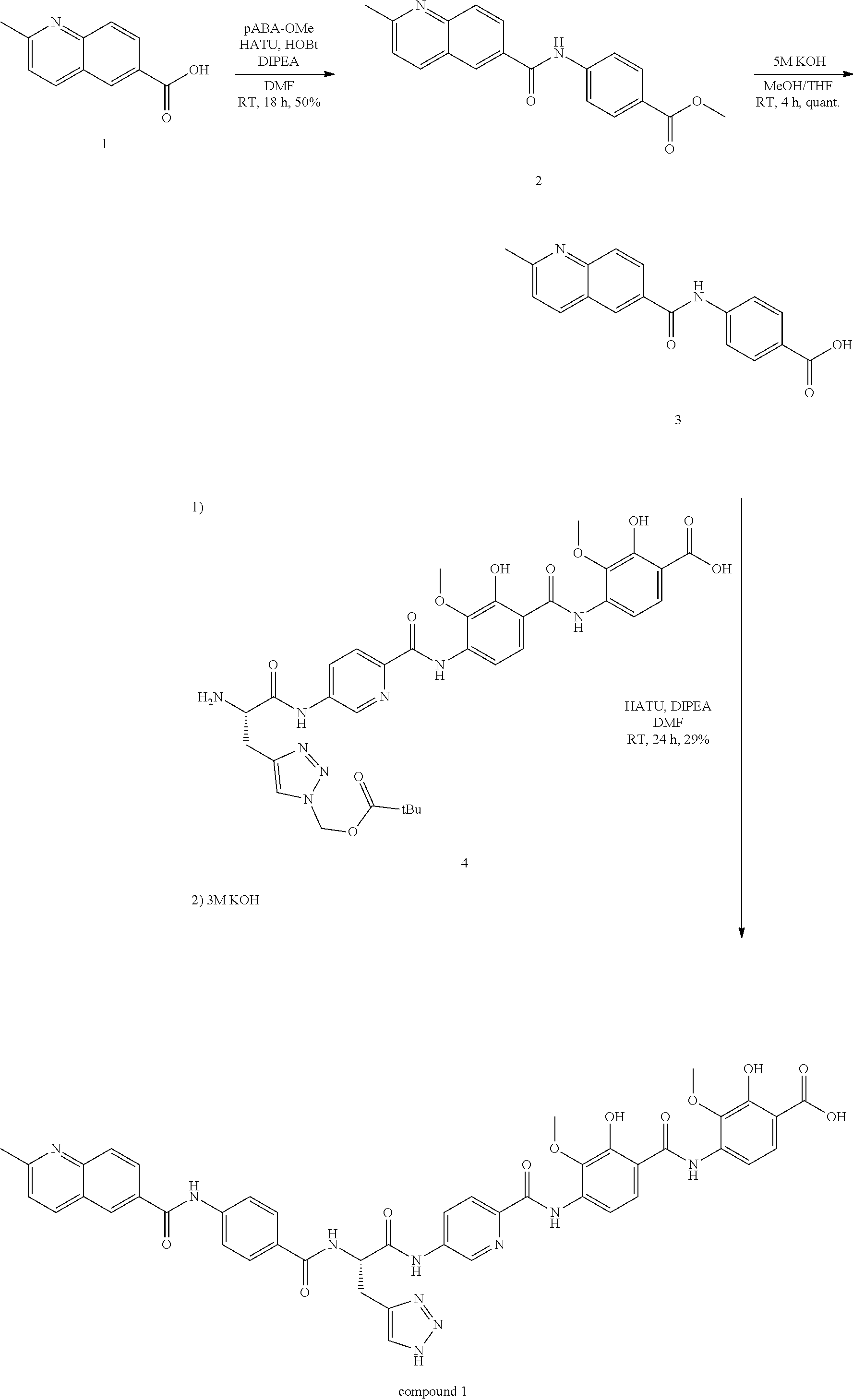

### Methyl 4-(2-methylquinoline-6-carboxamido)benzoate (2)

2-Methylquinoline-6-carboxylic acid (1, 100 mg, 534 μmol, 1.00 eq.) was dissolved in DMF (1 mL) and HOBt (36.1 mg, 267 μmol, 0.50 eq.), HATU (304 mg, 801 μmol, 1.50 eq.) and DIPEA (279 μL, 1.60 mmol, 3.00 eq.) were added and the reaction mixture was stirred for 1 h at room temperature. 4-Methyl-aminobenzoate (121 mg, 801 μmol, 1.50 eq.) was added and the reaction mixture was stirred 18 h at room temperature. The reaction mixture was diluted by EtOAc (30 mL) and the organic layer was washed by a saturated aqueous NaHCO₃ solution (3×20 mL) and by a saturated aqueous NaCl solution (1×20 mL). The organic layer was dried over MgSO₄, filtered and the solvent was removed under reduced pressure by rotary evaporation. The crude material was purified by flash column chromatography (SiO₂, EtOAc/Hex 1:1) and afforded methyl 4-(2-methylquinoline-6-carboxamido)benzoate (2, 85.0 mg, 267 μmol, 50%) as a white solid. ¹H NMR (400 MHz, DMSO-d₆) □ ppm 2.71 (s, 3H) 3.85 (s, 3H) 7.54 (d, J=8.53 Hz, 1H) 7.99 (s, 4H) 8.04 (d, J=8.78 Hz, 1H) 8.22 (dd, J=8.78, 2.01 Hz, 1H) 8.42 (d, J=8.53 Hz, 1H) 8.60 (d, J=1.76 Hz, 1H) 10.77 (s, 1H)¹³C NMR (DMSO-d₆, 101 MHz): □=165.8, 165.6, 161.0, 148.5, 143.6, 137.1, 131.5, 130.1, 128.4, 128.0, 125.3, 124.4, 123.1, 119.6, 51.9, 38.2, 25.0 ppm. HRMS (ESI): m/z calculated for C₁₉H₁₆N₂O₃ (M+H)⁺: 321.1229, found 321.1234.

### 4-(2-Methylquinoline-6-carboxamido)benzoic acid (3)

Methyl 4-(2-methylquinoline-6-carboxamido)benzoate (85.0 mg, 267 μmol, 1.00 eq.) was dissolved in MeOH/THF (2 mL, 1:1) and 5 M KOH solution (1 mL) was added and the reaction mixture was stirred for 18 h at room temperature. The volatiles were removed under reduced pressure by rotary evaporation and a 3 M HCl solution (2 mL) was added. The precipitated solid was filtered and washed by 1 M HCl solution. After drying at high vacuum 4-(2-methylquinoline-6-carboxamido)benzoic acid (3, 81.0 mg, 267 μmol, 100%) was obtained as brownish solid. HRMS (ESI): m/z calculated for C₁₈H₁₄N₂O₃ (M+H)⁺: 307.1074, found 307.1077.

### Compound 1

HATU (67.0 mg, 176 μmol, 1.35 eq.) was added to a solution of AB building block 3 (51.9 mg, 169 μmol, 1.30 eq.) in anhydrous DMF (1 mL) and the resulting solution was stirred at r.t for 45 min. A solution of tetrapeptide 4 (L. Zborovsky, L. Kleebauer, M. Seidel, A. Kostenko, L. von Eckardstein, F. O. Gombert, J. Weston, R. D. Süssmuth, Chem. Sci. 2021, 12, 14606-14617) (94.0 mg, 130 μmol, 1.00 eq.) and DIPEA (136 μL, 780 μmol, 6.00 eq.) in anhydrous DMF (1 mL) was added dropwise and the reaction mixture was stirred at r.t for 16 h. All volatiles were removed in vacuo and the residue was taken up in a mixture of THF (1 mL) and MeOH (1 mL), and 3 N KOH(aq.) (1 mL) was added dropwise. After 45 min of stirring, 3 N HCl(aq.) (1.1 mL) was added and the resulting suspension was evaporated under reduced pressure. The crude material was dissolved in DMSO, centrifuged, and the supernatant purified by HPLC (PLRP-S column, CH3CN in H2O). The title compound 4 (13 mg, 9% over two steps) was obtained as a colourless solid. ¹H NMR (DMSO-d₆, 700 MHz): δ (ppm)=11.71 (br. s., 1H), 11.58 (br. s., 1H), 11.12 (s, 1H), 10.86 (s, 1H), 10.82 (s, 1H), 10.49 (s, 1H), 8.94-9.01 (m, 1H), 8.84 (d, J=7.5 Hz, 2H), 8.77 (s, 1H), 8.40 (d, J=8.8 Hz, 1H), 8.34 (dd, J=8.5, 2.1 Hz, 1H), 8.21 (d, J=8.5 Hz, 1H), 8.17 (d, J=8.8 Hz, 1H), 8.11 (d, J=8.8 Hz, 1H), 8.03 (d, J=9.0 Hz, 1H), 7.92-7.98 (m, 4H), 7.88 (d, J=9.0 Hz, 1H), 7.82 (d, J=8.1 Hz, 1H), 7.72 (br. s., 1H), 7.59 (d, J=8.8 Hz, 1H), 4.96 (br. s., 2H), 3.92 (s, 3H), 3.88 (s, 3H), 3.34 (dd, J=14.8, 5.7 Hz, 1H), 3.28 (dd, J=14.8, 9.3 Hz, 1H), 2.86 ppm (s, 3H) H,C-HSQC NMR (DMSO-d₆, 101 MHz): S (ppm)=172.4, 171.6, 171.6, 171.6, 171.5, 166.4, 166.4, 165.3, 165.3, 165.2, 163.9, 163.8, 161.8, 160.9, 154.8, 150.0, 143.9, 143.8, 142.3, 140.0, 139.9, 139.9, 139.9, 139.3, 139.2, 138.2, 137.8, 137.7, 137.7, 136.6, 136.6, 136.6, 135.9, 133.6, 130.8, 129.1, 129.1, 129.0, 129.0, 127.6, 127.6, 127.6, 126.1, 126.1, 126.1, 124.3, 119.8, 119.8, 119.8, 119.8, 115.9, 115.9, 110.8, 110.8, 110.8, 110.7, 110.7, 110.7, 109.4, 60.6, 60.6, 54.7, 54.6, 39.9, 27.5, 23.4, 23.4 ppm. HRMS (ESI): m/z calculated for C₄₅H₃₈N₁₀O₁₁ (M+H)⁺: 895.2786, found 895.2794.

The following compounds are obtained in an analogous synthesis procedure.

### Compound 2

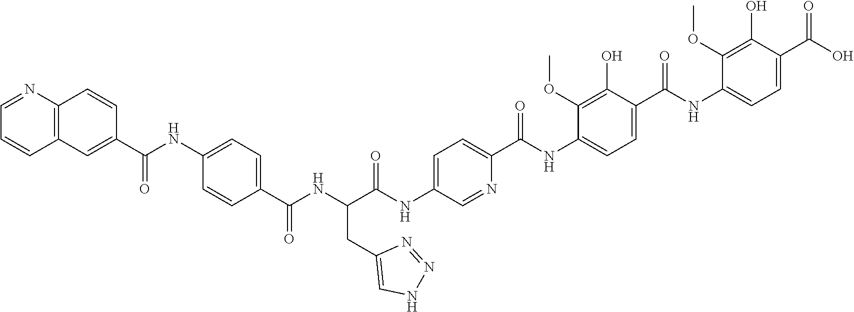 [SMILES: COc1c(NC(=O)c2ccc(NC(=O)c3ccc(NC(=O)C(Cc4c[nH]nn4)NC(=O)c4ccc(NC(=O)c5ccc6ncccc6c5)cc4)cn3)c(OC)c2O)ccc(C(=O)O)c1O]

¹H NMR (700 MHz, DMSO-d₆) δ (ppm) 11.73 (s, 1H), 11.60 (s, 1H), 11.14 (s, 1H), 10.87 (s, 1H), 10.50 (s, 1H), 9.10 (d, J=2.2 Hz, 1H), 8.99 (d, J=2.3 Hz, 1H), 8.85 (d, J=7.4 Hz, 1H), 8.73 (s, 1H), 8.68 (d, J=7.2 Hz, 1H), 8.35 (dd, J=8.6, 2.4 Hz, 1H), 8.33 (dd, J=8.6, 2.4 Hz, 1H), 8.22 (d, J=8.6 Hz, 1H), 8.20 (d, J=8.1, 1H), 8.13 (d, J=8.6, 1H), 8.04 (d, J=8.9 Hz, 1H), 7.98-7.90 (m, 4H), 7.89 (d, J=8.9 Hz, 1H), 7.75 (dd, J=8.1, 6.8, 1H), 7.73 (s, 1H), 7.60 (d, J=8.8 Hz, 1H), 4.96 (dd, J=7.5 Hz, 1H), 3.93 (s, 3H), 3.89 (s, 3H), 3.35 (dd, J=14.7, 5.7 Hz, 1H), 3.28 (dd, J=14.8, 9.2 Hz, 1H).

¹³C NMR (176 MHz, DMSO-d₆ from HSQC-ed) S (ppm) 152.4, 140.2, 139.6, 129.2, 129.1, 128.9, 128.7, 127.8, 127.0, 126.1, 123.4, 122.9, 119.9, 110.9, 110.7, 61.3, 60.6, 54.8, 27.7.

HRMS (ESI): m/z calced for C₄₄H₃₇N₁₀O₁₁ [M+H]⁺ 881.2636; found 881.2638.

### Compound 3

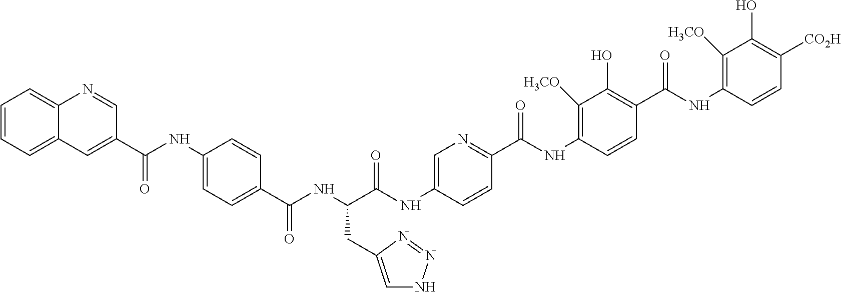 [SMILES: COc1c(NC(=O)c2ccc(NC(=O)c3ccc(NC(=O)[C@H](Cc4c[nH]nn4)NC(=O)c4ccc(NC(=O)c5cnc6ccccc6c5)cc4)cn3)c(OC)c2O)ccc(C(=O)O)c1O]

¹H NMR (700 MHz, DMSO-d6 from HSQC-ed) S (ppm) 11.73 (s, 1H), 11.60 (s, 1H), 11.14 (s, 1H), 10.86 (d, J=7.2 Hz, 2H), 10.50 (s, 1H), 9.39 (d, J=2.2 Hz, 1H), 9.00 (dd, J=17.0, 2.3 Hz, 2H), 8.85 (d, J=7.4 Hz, 1H), 8.35 (dd, J=8.6, 2.4 Hz, 1H), 8.22 (d, J=8.6 Hz, 1H), 8.18 (dd, J=8.1, 1.4 Hz, 1H), 8.17-8.10 (m, 2H), 8.04 (d, J=8.9 Hz, 1H), 7.98-7.90 (m, 5H), 7.89 (d, J=8.9 Hz, 1H), 7.75 (ddd, J=8.1, 6.8, 1.2 Hz, 1H), 7.72 (s, 1H), 7.66-7.50 (m, 3H), 4.96 (q, J=7.5 Hz, 1H), 3.93 (s, 3H), 3.88 (s, 3H), 3.35 (dd, J=14.7, 5.7 Hz, 1H), 3.28 (dd, J=14.8, 9.2 Hz, 1H).

¹³C NMR (176 MHz, DMSO from HSQC-ed) S (ppm) 149.51, 149.51, 139.99, 139.96, 136.76, 132.05, 132.03, 129.73, 129.26, 129.23, 128.92, 128.10, 127.74, 126.99, 126.14, 123.46, 119.87, 110.88, 110.77, 61.32, 60.68, 54.80.

HRMS (ESI): m/z calced for C₄₄H₃₇N₁₀O₁₁ [M+H]⁺ 881.2638; found 881.2642, tR=8.32 min.

### Compound 4

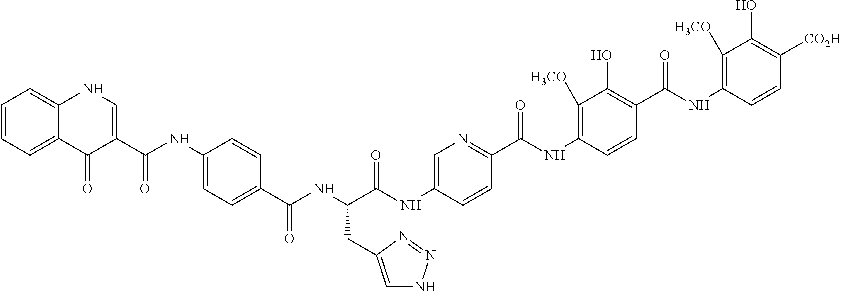 [SMILES: COc1c(NC(=O)c2ccc(NC(=O)c3ccc(NC(=O)[C@H](Cc4c[nH]nn4)NC(=O)c4ccc(NC(=O)c5c[nH]c6ccccc6c5=O)cc4)cn3)c(OC)c2O)ccc(C(=O)O)c1O]

¹H NMR (DMSO-d₆, 700 MHz): δ (ppm)=13.09 (br. s., 1H), 12.73 (s, 1H), 11.76 (br. s., 1H), 11.11 (s, 1H), 10.85 (s, 1H), 10.82 (s, 1H), 10.49 (s, 1H), 8.97 (s, 1H), 8.90 (d, J=6.1 Hz, 1H), 8.83 (br. s., 1H), 8.34 (d, J=8.4 Hz, 1H), 8.20 (d, J=8.4, 1H), 8.10 (d, J=8.8 Hz, 1H), 8.00 (d, J=8.4 Hz, 1H), 7.92 (d, J=8.4 Hz, 2H), 7.88-7.82 (m, 4H), 7.77 (br. s., J=8.2 Hz, 1H), 7.66 (br. s., 1H), 7.58-7.53 (m, 2H), 4.94 (dd, J¹=6.8 Hz, J²=13.5 Hz, 1H), 3.91 (s, 3H), 3.87 (s, 3H), 3.29-3.24 ppm (m, 2H).

H,C-HSQC NMR (DMSO-d₆, 101 MHz): S (ppm)=144.6, 140.0, 133.6, 133.4, 129.3, 127.7, 127.0, 126.0, 125.9, 123.5, 119.7, 119.2, 119.1, 110.7, 110.6, 61.3, 60.8, 54.8, 27.8 ppm.

HRMS (ESI): m/z calculated for C₄₅H₃₈N₁₀O₁₁ (M+H)⁺: 897.2509, found 897.2563.

### Compound 5:

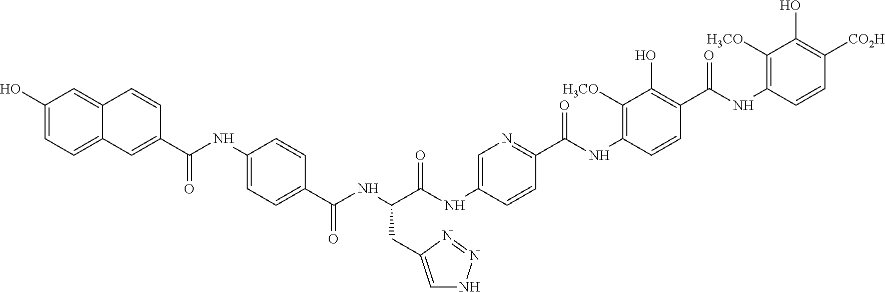 [SMILES: COc1c(NC(=O)c2ccc(NC(=O)c3ccc(NC(=O)[C@H](Cc4c[nH]nn4)NC(=O)c4ccc(NC(=O)c5ccc6cc(O)ccc6c5)cc4)cn3)c(OC)c2O)ccc(C(=O)O)c1O]

¹H NMR (700 MHz, DMSO-d₆) δ 14.66 (s, 3H), 11.72 (s, 3H), 11.13 (s, 3H), 10.85 (s, 5H), 10.51 (d, J=16.9 Hz, 8H), 10.11 (s, 4H), 8.81 (s, 4H), 8.72 (s, 1H), 8.50 (d, J=1.8 Hz, 4H), 8.35 (dd, J=8.5, 2.4 Hz, 4H), 8.22 (d, J=8.6 Hz, 4H), 8.12 (d, J=8.9 Hz, 4H), 8.03 (d, J=8.8 Hz, 3H), 7.95 (d, J=3.7 Hz, 4H), 7.96-7.90 (m, 21H), 7.89 (d, J=8.8 Hz, 3H), 7.82 (d, J=8.7 Hz, 4H), 7.77 (s, OH), 7.68 (s, 4H), 7.60 (d, J=8.8 Hz, 3H), 7.23-7.18 (m, 8H), 7.16 (s, 1H), 7.09 (s, 1H), 7.02 (s, 1H), 4.95 (d, J=7.6 Hz, 5H), 3.90 (d, J=6.5 Hz, 1H), 3.88 (s, 11H).

¹³C NMR (176 MHz, DMSO from HSQC-ed) δ 139.95, 131.32, 128.83, 128.70, 127.63, 126.99, 126.61, 126.16, 125.14, 123.45, 120.05, 119.73, 110.74, 109.15, 63.80, 61.26, 60.69.

HRMS (ESI): m/z calced for C₄₅H₃₈N₉O₁₂ [M+H]⁺ 896.2634; found 896.2643, tR=8.38 min

### Compound 6

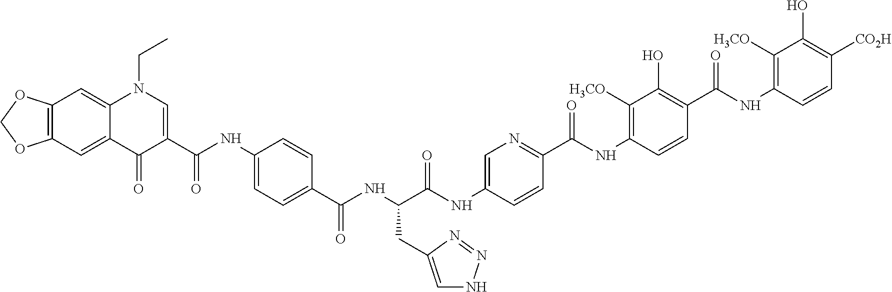 [SMILES: CCn1cc(C(=O)Nc2ccc(C(=O)N[C@@H](Cc3c[nH]nn3)C(=O)Nc3ccc(C(=O)Nc4ccc(C(=O)Nc5ccc(C(=O)O)c(O)c5OC)c(O)c4OC)nc3)cc2)c(=O)c2cc3c(cc21)OCO3]

¹H NMR (DMSO-d₆, 700 MHz): δ (ppm)=12.88 (s, 1H), 11.72 (br. s., 1H), 11.63 (br. s., 1H), 11.58 (br. s., 1H), 11.13 (s, 1H), 10.84 (s, 1H), 10.50 (s, 1H), 8.98 (m, 1H), 8.90 (s, 1H), 8.83 (br. s., 1H), 8.34 (d, J=8.7, 1H), 8.21 (d, J=8.6 Hz, 1H), 8.12 (d, J=8.9 Hz, 1H), 8.03 (d, J=8.9 Hz, 1H), 7.93 (d, J=8.6 Hz, 2H), 7.89 (d, J=8.9 Hz, 1H), 7.83 (d, J=8.7 Hz, 2H), 7.72 (s, 1H), 7.67 (br. s., 1H), 7.60-7.59 (m, 2H), 6.28 (s, 1H), 4.94 (dd, J¹=7.0 Hz, J²=14.6, Hz, 1H), 4.53 (q, J=7.1 Hz, 2H), 3.92 (s, 3H), 3.88 (s, 3H), 3.34 (dd, J¹=14.8, J²=5.7 Hz, 1H), 3.29-3.24 (m, 2H), 1.40 ppm (t, J=7.1 Hz, 3H).

H,C-HSQC NMR (DMSO-d₆, 101 MHz): S (ppm)=147.1, 140.0, 129.3, 127.6, 127.1, 126.1, 123.6, 119.2, 110.8, 110.6, 103.4, 103.0, 97.4, 61.4, 60.7, 54.7, 49.4, 27.8, 15.1 ppm.

HRMS (ESI): m/z calculated for C₄₅H₃₈N₁₀O₁₁ (M+H)⁺: 969.2786, found 969.2798.

### Compound 7

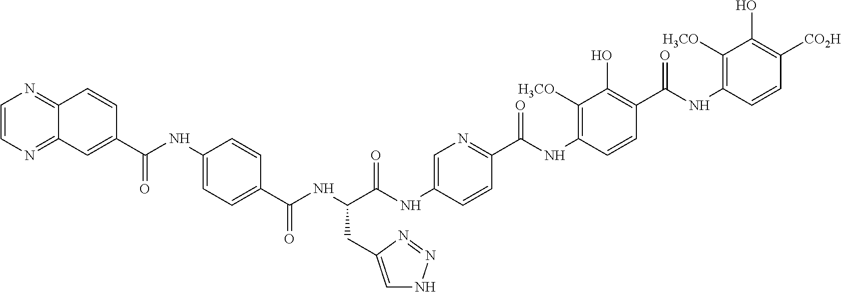 [SMILES: COc1c(NC(=O)c2ccc(NC(=O)c3ccc(NC(=O)[C@H](Cc4c[nH]nn4)NC(=O)c4ccc(NC(=O)c5ccc6nccnc6c5)cc4)cn3)c(OC)c2O)ccc(C(=O)O)c1O]

¹H NMR (DMSO-d₆, 700 MHz): δ (ppm)=11.71 (s, 1H), 11.58 (br. s., 1H), 11.14 (s, 1H), 10.87 (s, 1H), 10.86 (s, 1H), 10.50 (s, 1H), 9.10 (d, J=1.7 Hz, 1H), 9.08 (d, J=1.7 Hz, 1H), 8.99 (s, 1H), 8.85 (d, J=7.5 Hz, 1H), 8.80 (s, 1H), 8.38 (d, J=8.7 Hz, 1H), 8.35 (d, J=8.6, 1H), 8.26 (d, J=8.8 Hz, 1H), 8.22 (d, J=8.8 Hz, 1H), 8.12 (d, J=8.9 Hz, 1H), 8.04 (d, J=8.8 Hz, 1H), 7.98-7.95 (m, 4H), 7.89 (d, J=8.7 Hz, 1H), 7.73 (br. s., 1H), 7.60 (d, J=8.8 Hz, 1H), 4.96 (dd, J¹=7.5 Hz, J²=14.7 Hz, 1H), 3.93 (s, 3H), 3.89 (s, 3H), 3.35 (dd, J¹=5.9 Hz, J²=14.9 Hz, 1H), 3.28 (dd, J¹=9.0 Hz, J²=14.6 Hz, 1H) H,C-HSQC NMR (DMSO-d₆, 101 MHz): S (ppm)=147.5, 140.0, 130.0, 129.5, 129.4, 128.9, 127.7, 126.1, 123.4, 120.0, 110.9, 110.7, 61.3, 60.8, 54.8, 27.7.

HRMS (ESI): m/z calculated for C₄₅H₃₈N₁₀O₁₁ (M+H)⁺: 882.2590, found 882.2588.

### Compound 8

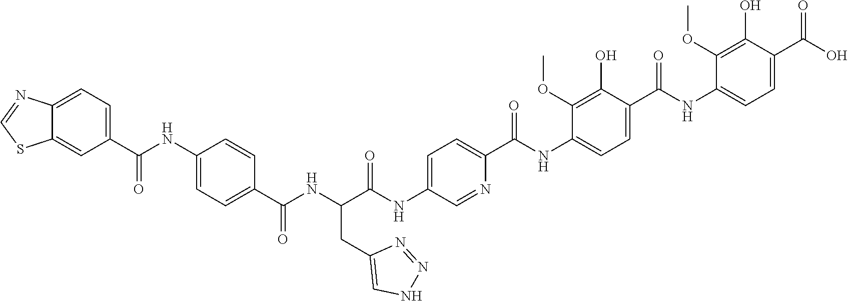 [SMILES: COc1c(NC(=O)c2ccc(NC(=O)c3ccc(NC(=O)C(Cc4c[nH]nn4)NC(=O)c4ccc(NC(=O)c5ccc6ncsc6c5)cc4)cn3)c(OC)c2O)ccc(C(=O)O)c1O]

¹H NMR (700 MHz, DMSO-d₆) δ (ppm) 11.73 (s, 1H), 11.60 (s, 1H), 11.14 (s, 1H), 10.86 (s, 1H), 10.50 (s, 1H), 9.60 (s, 1H), 8.99 (d, J=2.3 Hz, 1H), 8.84 (d, J=7.4 Hz, 1H), 8.82 (d, J=2.3 Hz, 1H), 8.73 (s, 1H), 8.35 (dd, J=8.6, 2.4 Hz, 1H), 8.25 (d, J=8.6 Hz, 1H), 8.22 (d, J=8.6 Hz, 1H), 8.14 (d, J=8.1, 1H), 8.13 (d, J=8.6, 1H), 8.04 (d, J=8.9 Hz, 1H), 7.97-7.90 (m, 4H), 7.89 (d, J=8.9 Hz, 1H), 7.72 (s, 1H), 7.60 (d, J=8.8 Hz, 1H), 4.96 (dd, J=7.5 Hz, 1H), 3.93 (s, 3H), 3.88 (s, 3H), 3.35 (dd, J=14.7, 5.7 Hz, 1H), 3.28 (dd, J=14.8, 9.2 Hz, 1H).

¹³C NMR (176 MHz, DMSO-d₆ from HSQC-ed) S (ppm) 159.6, 140.0, 128.9, 127.7, 127.0, 126.3, 126.1, 123.4, 123.3, 119.8, 110.9, 110.7, 61.3, 60.7, 54.8, 27.6.

.HRMS (ESI): m/z calcd for C₄₄H₃₇N₁₀O₁₁ [M+H]⁺ 887.2192; found 887.2198.

### Compound 9

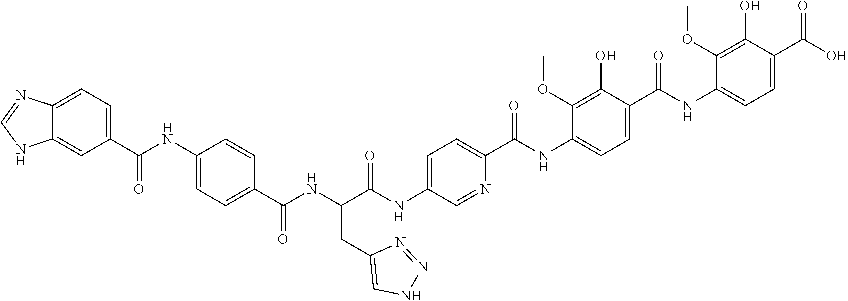 [SMILES: COc1c(NC(=O)c2ccc(NC(=O)c3ccc(NC(=O)C(Cc4c[nH]nn4)NC(=O)c4ccc(NC(=O)c5ccc6nc[nH]c6c5)cc4)cn3)c(OC)c2O)ccc(C(=O)O)c1O]

¹H NMR (700 MHz, DMSO-d₆) δ (ppm) 11.73 (s, 1H), 11.60 (s, 1H), 11.14 (s, 1H), 10.86 (s, 1H), 10.63 (s, 1H), 10.50 (s, 1H), 9.21 (s, 1H), 8.98 (d, J=2.3 Hz, 1H), 8.83 (d, J=7.4 Hz, 1H), 8.44 (s, 1H), 8.35 (dd, J=8.6, 2.4 Hz, 1H), 8.22 (d, J=8.6 Hz, 1H), 8.12 (d, J=8.6 Hz, 1H), 8.07 (d, J=7.3 Hz, 1H), 8.04 (d, J=8.9 Hz, 1H), 7.97-7.92 (m, 4H), 7.91 (d, J=8.9 Hz, 1H) 7.89 (d, J=8.9 Hz, 1H), 7.72 (s, 1H), 7.60 (d, J=8.8 Hz, 1H), 4.96 (dd, J=7.5 Hz, 1H), 3.93 (s, 3H), 3.88 (s, 3H), 3.35 (dd, J=14.7, 5.7 Hz, 1H), 3.28 (dd, J=14.8, 9.2 Hz, 1H).

¹³C NMR (176 MHz, DMSO-d₆ from HSQC-ed) S (ppm) 140.0, 128.9, 127.7, 127.1, 126.1, 124.7, 123.5, 119.8, 110.9, 110.7, 61.3, 60.8, 54.7, 27.6.

.HRMS (ESI): m/z calcd for C₄₄H₃₇N₁₀O₁₁ [M+H]⁺ 870.2591; found 870.2596.

### Compound 10

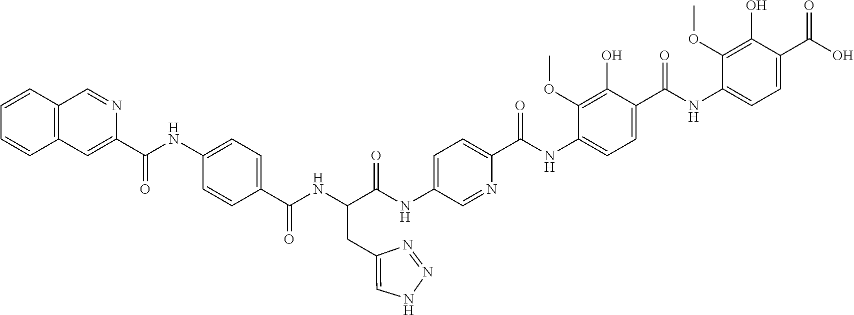 [SMILES: COc1c(NC(=O)c2ccc(NC(=O)c3ccc(NC(=O)C(Cc4c[nH]nn4)NC(=O)c4ccc(NC(=O)c5cc6ccccc6cn5)cc4)cn3)c(OC)c2O)ccc(C(=O)O)c1O]

¹H NMR (700 MHz, DMSO-d₆) δ (ppm) 11.73 (s, 1H), 11.60 (s, 1H), 11.14 (s, 1H), 11.00 (s, 1H), 10.87 (s, 1H), 10.50 (s, 1H), 9.50 (s, 1H), 8.99 (d, J=2.3 Hz, 1H), 8.85 (d, J=7.4 Hz, 1H), 8.75 (s, 1H), 8.35 (dd, J=8.6, 2.4 Hz, 1H), 8.33 (d, J=8.3 Hz, 1H), 8.28 (d, J=7.8 Hz, 1H), 8.12 (d, J=8.8, 1H), 8.10 (d, J=8.8, 2H), 8.04 (d, J=8.8 Hz, 1H), 7.95-7.92 (m, 3H), 7.90-7.86 (m, 2H), 7.73 (s, 1H), 7.60 (d, J=8.8 Hz, 1H), 4.96 (dd, J=7.5 Hz, 1H), 3.93 (s, 3H), 3.89 (s, 3H), 3.35 (dd, J=14.7, 5.7 Hz, 1H), 3.28 (dd, J=14.8, 9.2 Hz, 1H).

¹³C NMR (176 MHz, DMSO-d₆ from HSQC-ed) S (ppm) 152.1, 140.0, 132.2, 130.2, 128.9, 128.6, 128.5, 128.4, 127.0, 126.2, 123.4, 119.9, 110.9, 110.8, 61.3, 60.6, 54.8, 27.5.

HRMS (ESI): m/z calcd for C₄H₃₇N₁₀O₁₁ [M+H]⁺ 881.2636; found 881.2638.

### Compound 11

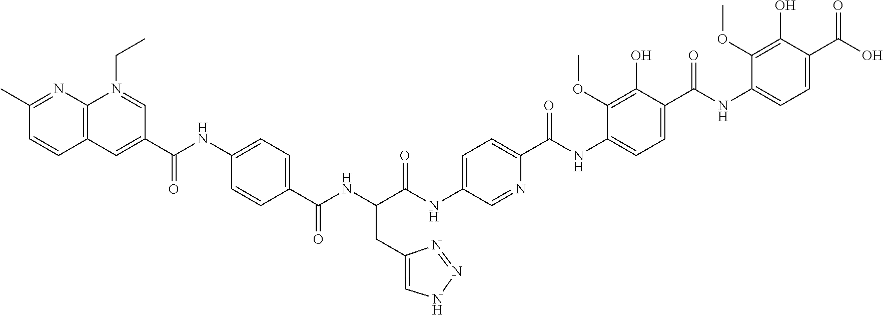

¹H NMR (500 MHz, DMSO-d₆ from HSQC-ed) S (ppm) 12.47 (s, 1H), 11.78 (s, 1H), 10.98 (s, 1H), 10.88 (s, 1H), 10.49 (s, 1H), 9.16 (s, 1H), 8.98 (s, 1H), 8.84 (s, 1H), 8.64 (d, 1H), 8.35 (dd, J=8.6, 2.4 Hz, 1H), 8.21 (d, J=8.1 Hz, 1H), 8.09 (d, J=8.6 Hz, 1H), 7.97-7.78 (m, 6H), 7.66 (s, 1H), 7.55 (d, J=8.0 Hz, 1H), 4.94 (dd, J=7.5 Hz, 1H), 4.65 (q, 2H), 3.90 (s, 3H), 3.88 (s, 3H), 3.35-3.24 (m, 2H), 1.44 (t, 3H).

¹³C NMR (176 MHz, DMSO-d₆ from HSQC-ed) S (ppm) 149.8, 140.1, 136.5, 129.3, 128.9, 127.8, 126.9, 123.5, 122.6, 119.2, 110.4, 109.7, 61.3, 60.6, 54.8, 47.1, 27.7, 15.1.

HRMS (ESI): m/z calcd for C₄H₃₇N₁₀O₁₁ [M+H]⁺ 940.3009; found 940.3004.

### Compound 12

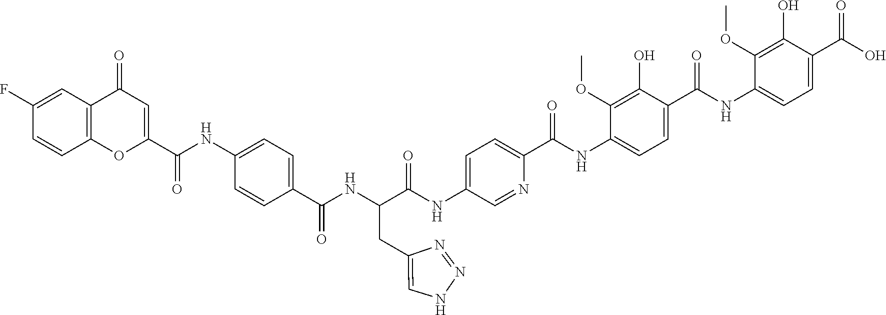 [SMILES: COc1c(NC(=O)c2ccc(NC(=O)c3ccc(NC(=O)C(Cc4c[nH]nn4)NC(=O)c4ccc(NC(=O)c5cc(=O)c6cc(F)ccc6o5)cc4)cn3)c(OC)c2O)ccc(C(=O)O)c1O]

¹H NMR (500 MHz, DMSO-d₆ from HSQC-ed) S (ppm) 11.74 (s, 1H), 11.10 (s, 1H), 11.06 (s, 1H), 10.89 (d, 1H), 10.48 (s, 1H), 8.98 (s, 1H), 8.89 (s, 1H), 8.34 (d, J=8.0, 1H), 8.20 (d, J=8.0 Hz, 1H), 8.10 (d, J=8.8 Hz 1H), 8.02-7.82 (m, 10H), 7.77 (d, J=7.2 Hz, 1H), 7.68 (s, 1H), 7.58 (d, J=8.0, 1H), 7.01 (s, 1H), 4.95 (m, 1H), 3.91 (s, 3H), 3.87 (s, 3H), 3.35-3.24 (m, 2H).

¹³C NMR (176 MHz, DMSO-d₆ from HSQC-ed) S (ppm) 140.0, 129.9, 127.9, 126.9, 126.1, 123.6, 123.4, 121.1, 120.8, 119.2, 111.0, 110.2, 61.3, 60.6, 54.9, 47.1, 27.8.

HRMS (ESI): m/z calcd for C₄H₃₇N₁₀O₁₁ [M+H]⁺ 916.2333; found 916.2321.

### Compound 13

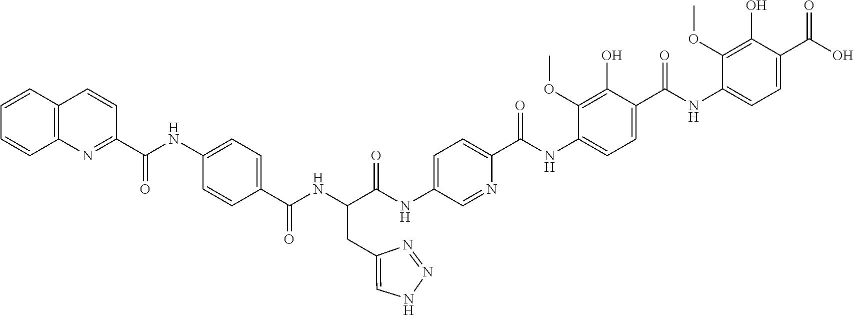 [SMILES: COc1c(NC(=O)c2ccc(NC(=O)c3ccc(NC(=O)C(Cc4c[nH]nn4)NC(=O)c4ccc(NC(=O)c5ccc6ccccc6n5)cc4)cn3)c(OC)c2O)ccc(C(=O)O)c1O]

¹H NMR (700 MHz, DMSO-d₆) δ (ppm) 11.73 (s, 1H), 11.60 (s, 1H), 11.14 (s, 1H), 10.98 (s, 1H), 10.86 (s, 1H), 10.50 (s, 1H), 8.99 (d, J=2.3 Hz, 1H), 8.87 (d, J=7.4 Hz, 1H), 8.67 (d, 1H), 8.68 (d, J=7.2 Hz, 1H), 8.35 (dd, J=8.6, 2.4 Hz, 1H), 8.29 (dd, J=8.6, 2.4 Hz, 1H), 8.27 (d, J=8.6 Hz, 1H), 8.22 (d, J=8.1, 1H), 8.15 (d, J=8.6, 1H), 8.12 (d, J=8.9 Hz, 1H), 8.10 (d, J=8.7 Hz, 2H), 8.04 (d, 1H), 7.97 (d, J=8.8 Hz, 2H), 7.94 (d, J=8.9 Hz, 1H), 7.89 (d, 1H), 7.79 (dd, J=8.1, 6.8, 1H), 7.73 (s, 1H), 7.60 (d, J=8.8 Hz, 1H), 4.96 (dd, J=7.5 Hz, 1H), 3.93 (s, 3H), 3.89 (s, 3H), 3.35 (dd, J=14.7, 5.7 Hz, 1H), 3.28 (dd, J=14.8, 9.2 Hz, 1H).

¹³C NMR (176 MHz, DMSO-d₆ from HSQC-ed) S (ppm) 140.0, 138.8, 131.1, 129.8, 129.0, 128.9, 128.8, 128,7 127.8, 127.0, 126.2, 123.5, 119.9, 119.3, 110.9, 110.7, 61.3, 60.6, 54.80, 27.7.

HRMS (ESI): m/z calcd for C₄₄H₃₇N₁₀O₁₁ [M+H]⁺ 881.2631; found 881.2636.

### Compound 14

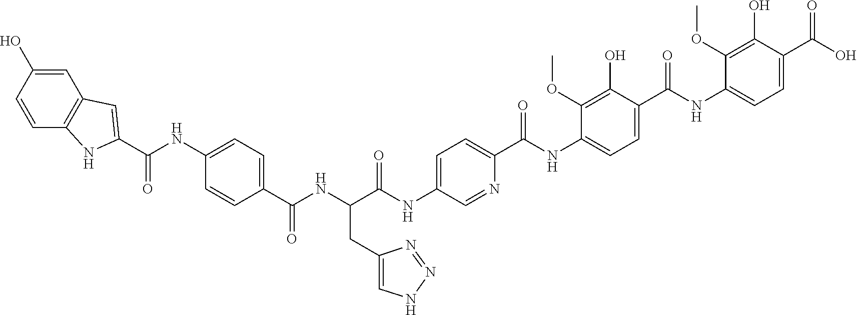 [SMILES: COc1c(NC(=O)c2ccc(NC(=O)c3ccc(NC(=O)C(Cc4c[nH]nn4)NC(=O)c4ccc(NC(=O)c5cc6cc(O)ccc6[nH]5)cc4)cn3)c(OC)c2O)ccc(C(=O)O)c1O]

¹H NMR (700 MHz, DMSO-d₆) δ (ppm) 11.39-11.36 (m, 2H), 10.87-10.84 (m, 3H), 10.48 (s, 1H), 10.26 (s, 1H), 9.32 (s, 1H), 8.97 (d, J=2.5 Hz, 1H), 8.79 (d, J=7.6 Hz, 1H), 8.39 (s, 3H), 8.35 (dd, J=8.5, 2.4 Hz, 1H), 8.21 (d, J=8.5 Hz, 1H), 8.02 (s, 1H), 7.96 (s, 2H), 7.93-7.90 (m, 9H), 7.84 (d, J=7.9 Hz, 2H), 7.72 (s, 2H), 7.54 (s, 1H), 7.47 (d, J=8.6 Hz, 1H), 7.40 (d, J=8.8 Hz, 1H), 7.37 (d, J=2.4 Hz, 1H), 7.29-7.25 (m, 1H), 7.24-7.19 (m, 2H), 6.82 (d, J=2.1 Hz, 1H), 6.62 (dd, J=8.6, 2.2 Hz, 1H), 6.56 (s, 1H), 4.94 (m, 1H), 3.89 (s, 3H), 3.86 (s, 3H)

¹³C NMR (176 MHz, DMSO from HSQC-ed) S (ppm) 162.7, 139.95, 128.9, 128.8, 127.9, 127.6, 126.2, 124.90, 123.3, 123.0, 119.4, 112.2, 108.3, 105.3, 96.91, 61.0, 59.8, 54.73.

HRMS (ESI): m/z calcd for C₄₃H₃₇N₁₀O₁₂ [M+H]⁺ 885.2587; found 885.2590.

### Compound 15

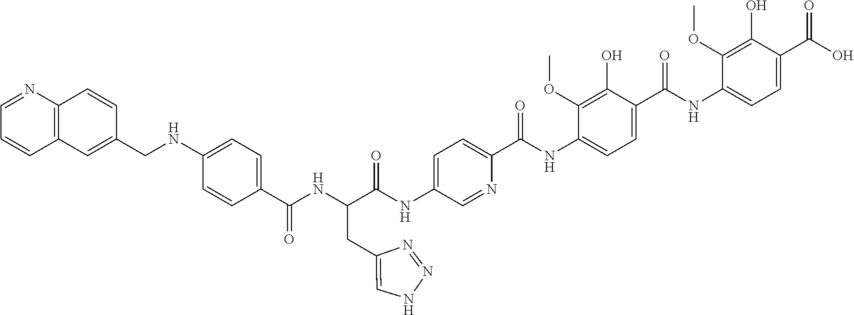 [SMILES: COc1c(NC(=O)c2ccc(NC(=O)c3ccc(NC(=O)C(Cc4c[nH]nn4)NC(=O)c4ccc(NCc5ccc6ncccc6c5)cc4)cn3)c(OC)c2O)ccc(C(=O)O)c1O]

¹H NMR (700 MHz, DMSO-d₆) δ (ppm) 11.72 (s, 1H), 11.60 (s, 1H), 11.13 (s, 1H), 10.78 (s, 1H), 10.48 (s, 1H), 9.14 (d, J=4.5 Hz, 1H), 8.95 (d, J=2.5 Hz, 1H), 8.87 (d, J=7.9 Hz, 1H), 8.38 (dd, J=8.6, 2.4 Hz, 1H), 8.31 (dd, J=8.6, 2.3 Hz, 1H), 8.19 (d, J=8.5 Hz, 1H), 8.17 (d, J=8.7, 1H), 8.14 (s, 1H), 8.11 (d, J=8.9 Hz, 1H), 8.03 (d, J=8.8 Hz, 1H), 8.01 (dd, J=8.6, 1.9 Hz, 1H), 7.90-7.87 (m, 4H), 7.67-7.64 (m, 3H), 7.60 (d, J=9.0 Hz, 1H), 6.64 (d, J=8.9, 2H), 4.85 (dd, J=7.8, 6.2 Hz, 1H), 4.62 (s, 1H), 3.92 (s, 3H), 3.87 (s, 3H), 3.27 (dd, J=15.1, 5.8 Hz, 1H), 3.21 (dd, J=15.0, 9.2 Hz, 1H).

¹³C NMR (176 MHz, DMSO-d₆ from HSQC-ed) S (ppm) 157.6, 147.7, 140.0, 132.9, 129.6, 127.6, 127.0, 126.3, 126.0, 124.6, 122.5, 111.7, 110.9, 110.7, 61.3, 60.6, 54.6, 46.2, 27.6.

HRMS (ESI): m/z calcd for C₄₄H₃₉N₁₀O₁₀ [M+H]⁺ 867.2838; found 867.2845.

### Compound 16

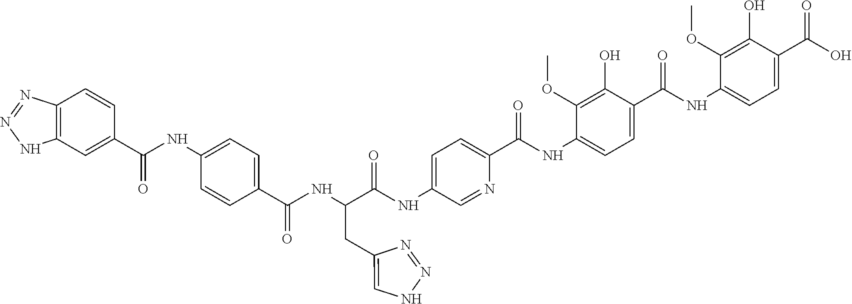 [SMILES: COc1c(NC(=O)c2ccc(NC(=O)c3ccc(NC(=O)C(Cc4c[nH]nn4)NC(=O)c4ccc(NC(=O)c5ccc6nn[nH]c6c5)cc4)cn3)c(OC)c2O)ccc(C(=O)O)c1O]

¹H NMR (700 MHz, DMSO-d₆) δ (ppm) 11.73 (s, 1H), 11.60 (s, 1H), 11.14 (s, 1H), 10.86 (s, 1H), 10.66 (s, 1H), 10.50 (s, 1H), 8.98 (d, J=2.5 Hz, 1H), 8.84 (d, J=7.9 Hz, 1H), 8.35 (dd, J=8.6, 2.4 Hz, 1H), 8.22 (d, J=8.5 Hz, 1H), 8.12 (d, J=8.7, 1H), 8.10-8.01 (m, 3H), 7.97-7.90 (m, 4H), 7.73 (s, 1H), 7.60 (d, J=9.0 Hz, 1H), 4.96 (dd, J=7.8, 6.2 Hz, 1H), 3.93 (s, 3H), 3.88 (s, 3H), 3.35 (dd, J=15.1, 5.8 Hz, 1H), 3.29 (dd, J=15.0, 9.2 Hz, 1H).

¹³C NMR (176 MHz, DMSO-d₆ from HSQC-ed) S (ppm) 139.8, 134.9, 128.8, 127.8, 127.0, 126.2, 123.3, 119.8, 110.9, 110.7, 61.3, 60.6, 54.6, 46.2, 27.6.

HRMS (ESI): m/z calcd for C₄₁H₃₅N₁₂O₁₁ [M+H]⁺ 871.2541; found 871.2535.

### Test for Biological Activity

### Strains:

E. coli DSM 1116; E. coli BW25113, S. typhimurium TA100; Bacillus subtilis DSM10; Micrococcus luteus DSM1790 and M. phlei DSM 750

### Biological Testing:

The tests were performed using the micro dilution method.

### Microdilution Assay:

The determination of MIC values was performed according to the ninth edition of the Approved Standard M07-A9 (CLSI. Methods for Dilution Antimicrobial Susceptibility Tests for Bacteria That Grow Aerobically; Approved Standard-Ninth Edition. CLSI document M07-A9. Wayne, PA: Clinical and Laboratory Standards Institute; 2012.)

The test was carried out for different bacterial strains (E. coli DSM 1116, E. coli BW25113 [gram negative], B. subtilis DSM 10 [gram positive], M. luteus DSM 1790 [gram positive], S. typhimurium TA100 [gram negative]) and M. phlei DSM 750. 20 μL of cryo stock of each strain were inoculated in 20 mL of LB media (Lysogeny broth: 10 g/L peptone, 5 g/L yeast extract, 5 g/L NaCl) followed by incubation over night at 37° C., 200 rpm. The test inoculum was adjusted by the 0.5 McFarland Standard (OD625 from 0.08 to 0.1). Within 15 min of preparation, the adjusted inoculum suspension was diluted in MHBII media (BBL TM Mueller-Hinton Broth II, Becton, Dickinson and Company, New Jersey/USA) so that each well contained approximately 5×105 CFU/mL in a final volume of 100 μL. 95 μL of the inoculum were applied per well and 5 μL of the (diluted) antibiotic substance were added.

Previously the dry antibiotic compounds were dissolved in DMSO (100%) with a concentration of 2560 μg/mL and the resulting stock solutions were further diluted in DMSO (100%). 5 μL of each antibiotic dilution were applied to the microdilution tray to reach final concentrations of 64 μg/mL to 0.008 μg/mL. One row of each well plate was left as a growth control without antibiotic substances and another row of the microdilution tray was used as sterility control (only MHB II-media). The antimicrobial effect of the solvent (DMSO) was tested by adding 5 μL DMSO to several wells without antibiotics. Purity check and cell titer control were performed according to International Standard M07-A9 on Mueller-Hinton II Agar (Mueller Hinton II Broth, 15 g/L agar-agar). Both microdilution trays and agar plates were incubated at 37° C. for 20 h and subsequently analyzed visually.

### Retention Time

The product was dissolved in DMSO, centrifuged, and the supernatant injected into an HPLC (PLRP-S column, CH₃CN in H₂O). RT (Retention Time) is measured in minutes.

### Clog P

The clogP was calculated using the Chemdraw software

### MIC Determination (Medium: MHBII/Human Serum (1:1))

The test was carried out for bacterial strain, E. coli BW25113 (Gram-negative).

First, 20 μl of glycerol stock of the strain was inoculated in 20 mL LB followed by incubation overnight at 37° C., 200 r.p.m.

The serum was subjected to a heat shock (56° C./30 min in a water bath) before use.

The test inoculum was adjusted by the 0.5 McFarland Standard, so that each well contained approximately 5×10⁵ c.f.u. mL⁻¹ in a final volume of 100 μL. For the adjustment of the test inoculum, MHBII medium and the human serum were used in a mixing ratio of 1:1.

For dilutions, the dry powders were dissolved in DMSO with a concentration of 2.56 mg mL-1 and serially diluted in DMSO (100%). Initial solution and serial dilutions of Ciprofloxacin were prepared in 0.1 N HCl.

Then, 95 μL of the adjusted inoculum was applied per well and 5 μl of each antibiotic dilution was applied to the microdilution tray to reach final concentrations of 8 to 0.016 μg ml⁻¹.

The antimicrobial effect of the solvent (DMSO or 0.1 N HCL) was tested by adding 5 μL DMSO or 0.1 N HCL to several wells.

One row of each well plate served as a growth control without antibiotic substances, and another row of the microdilution tray served as sterility control (MHB II-media).

The MIC testing results and RT results for compounds 1-14 are summarized in table 1. The MIC data for compounds 15 and 16 are provided separately.

The potency of an antibiotic is determined by the minimum inhibitory concentration (MIC). Contrary to intuition, a particularly low value is equated with a high potency.

The 3^{rd} generation Albicidin is used as reference compound and is of the following structure:

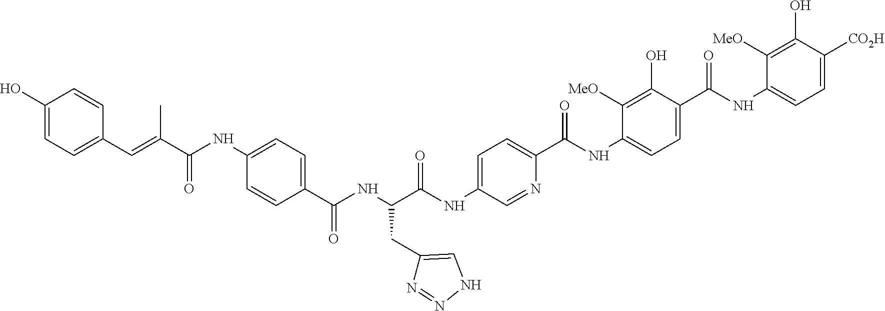

TABLE 1
Antibacterial activity of compounds according to the solution against selected strains

| Structure | | | | | | |
|---|---|---|---|---|---|---|
| A building block | | | | | | |
| Strain | 3^{RD} GEN Alb i | | | | | |
| | | 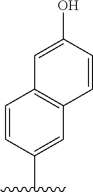 [SMILES: *c1ccc2cc(O)ccc2c1] | 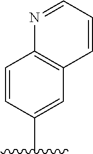 [SMILES: *c1ccc2ncccc2c1] | 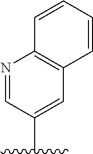 [SMILES: *c1cnc2ccccc2c1] | 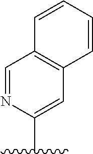 [SMILES: *c1cc2ccccc2cn1] | 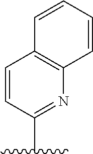 [SMILES: *c1ccc2ccccc2n1] |
| | | 5 | 2 | 3 | 10 | 13 |
| E. coli BW25113 | 0.016 | ≤0.016 | ≤0.016 | ≤0.016 | ≤0.016 | ≤0.016 |
| E. coli DSM1116 | 0.016 | 0.016 | 0.016 | 0.031 | ≤0.016 | ≤0.016 |
| S. typhimirium | 0.016 | ≤0.016 | ≤0.016 | ≤0.016 | ≤0.016 | ≤0.016 |
| B. subtilis | 0.031 | 0.063 | 0.25 | 0.125 | 0.063 | ≤0.016 |
| M. luteus | 0.031 | 0.031 | 0.125 | 0.125 | 0.063 | ≤0.016 |
| M. phlei | 0.25 | 1.00 | 4.00 | 1.00 | 1.00 | 0.5 |
| E. coli + | 0.5 | 2.00 | 4.00 | 2.00 | 1.00 | 2.00 |
| 50% human | | | | | | |
| serum | | | | | | |
| RT (min) | 8.15 | 8.15 | 7.67 | 8.07 | 8.74 | 9.49 |
| | | 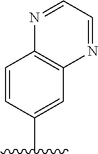 [SMILES: *c1ccc2nccnc2c1] | 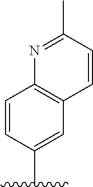 [SMILES: *c1ccc2nc(C)ccc2c1] | 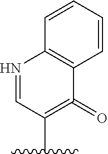 | 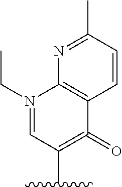 | 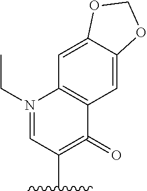 |
| | | 7 | 1 | 4 | 11 | 6 |
| E. coli BW25113 | 0.016 | 0.031 | 0.016 | 0.063 | 0.063 | ≤0.016 |
| E. coli DSM1116 | 0.016 | 0.031 | 0.016 | 0.031 | 0.031 | 0.016 |
| S. typhimirium | 0.016 | ≤0.016 | ≤0.016 | ≤0.016 | ≤0.016 | ≤0.016 |
| B. subtilis | 0.031 | 0.125 | 0.063 | ≤0.016 | 0.063 | 0.031 |
| M. luteus | 0.031 | 0.25 | 0.063 | ≤0.016 | 0.031 | 0.016 |
| M. phlei | 0.25 | 2.00 | 0.5 | 8.00 | ≥8 | 0.25 |
| E. coli + | 0.5 | 1.00 | 0.5 | 2.00 | 8.00 | 0.5 |
| 50% human | | | | | | |
| serum | | | | | | |
| RT (min) | 8.15 | 7.71 | 6.94 | 7.98 | 9.24 | 8.45 |
| | | 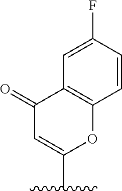 [SMILES: *c1cc(=O)c2cc(F)ccc2o1] | 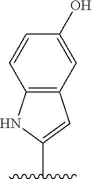 [SMILES: *c1cc2cc(O)ccc2[nH]1] | 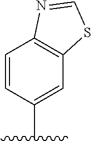 [SMILES: *c1ccc2ncsc2c1] | 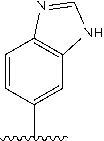 [SMILES: *c1ccc2nc[nH]c2c1] | |
| | | 12 | 14 | 8 | 9 | |
| E. coli BW25113 | 0.016 | ≤0.016 | 0.125 | ≤0.016 | 0.125 | |
| E. coli DSM1116 | 0.016 | ≤0.016 | 0.063 | ≤0.016 | 0.125 | |
| S. typhimirium | 0.016 | ≤0.016 | 0.125 | ≤0.016 | 0.125 | |
| B. subtilis | 0.031 | 0.125 | 1.00 | 0.125 | 2.00 | |
| M. luteus | 0.031 | 0.063 | 1.00 | 0.125 | 2.00 | |
| M. phlei | 0.25 | 2.00 | 4.00 | 2.00 | 8.00 | |
| E. coli + | 0.5 | 8.00 | ≥8 | 0.5 | ≥8 | |
| 50% human | | | | | | |
| serum | | | | | | |
| RT (min) | 8.15 | 8.60 | 7.50 | 7.65 | 6.69 | |

### Compound 15:

| | |
|---|---|
| MIC values against E. coli BW25113 | 0.031 |
| MIC values against E. coli DSM 1116 | 0.063 |
| MIC values against B. subtilis DSM10 | 0.25-0.5 |
| MIC values against M. luteus DSM 1790 | 0.125 |
| MIC values against M. phlei DSM 750 | 2.0 |
| MIC values against S. typhimurium TA100 | 0.031 |
| MIC values against E. coli BW25113 human serum | 4.0-8.0 |

### Compound 16:

| | |
|---|---|
| MIC values against E. coli BW25113 | 0.031 |
| MIC values against E. coli DSM 1116 | 0.063 |
| MIC values against B. subtilis DSM10 | 0.5-1.0 |
| MIC values against M. luteus DSM 1790 | 1.0 |
| MIC values against M. phlei DSM 750 | 4.0-8.0 |
| MIC values against S. typhimurium TA100 | 0.125 |
| MIC values against E. coli BW25113 human serum | 8.0 |

1. A compound having the general formula (1) 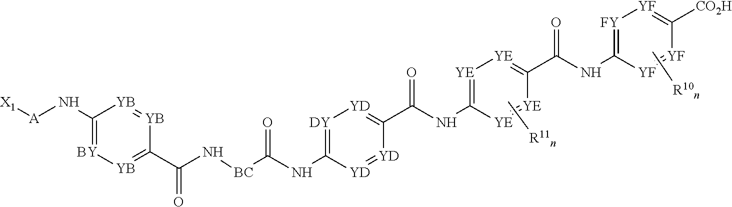
with A being CH₂ or CO, preferably CO,
with X₁ being
a substituted or unsubstituted 9-10 membered bicyclic system with both rings aromatic or one ring being aromatic and the other ring containing at least one double bond or one ring being aromatic and the other ring being alicyclic,
a substituted or unsubstituted a 9-10 membered bicyclic heterocyclic system with both rings aromatic or one ring being aromatic and the other ring containing at least one double bond or one ring being aromatic and the other ring being alicyclic, wherein at least one heteroatom is N, S or O,
b) with BC being 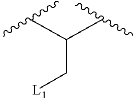
with L₁ being a substituted or unsubstituted C₅-C₆ aromatic heterocycle,
c) with n of R¹⁰ₙ and n of R¹¹ₙ being independently from each other 0, 1, 2, 3 or 4, in particular n of R¹⁰ₙ and n of R¹¹ₙ being 0, 1, 2 or 3, more particular n of R¹⁰ₙ and n of R¹¹ₙ being 1 and 2; with each R¹⁰ and R¹¹ being selected independently from any other R¹⁰ and R¹¹ from —OH, —F, —Cl, —Br, —I, —CCH, —CN, —OC₁-C₆ alkyl, in particular from —OH, —F, —OCH₃, —OC₂H₅, -OiC₃H₇, -OnC₃H₇, —OCF₃; and
d) with YB, YD, YE and YF being independently from each other N, CF, CCl or CH, in particular N and CH.

2. The compound according to claim 1, wherein X₁ is one of the following:
Naphthalenes and indenes 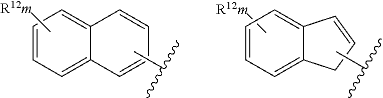
Indoles and Isoindoles 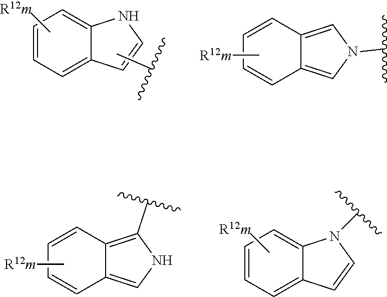
Benzazoles 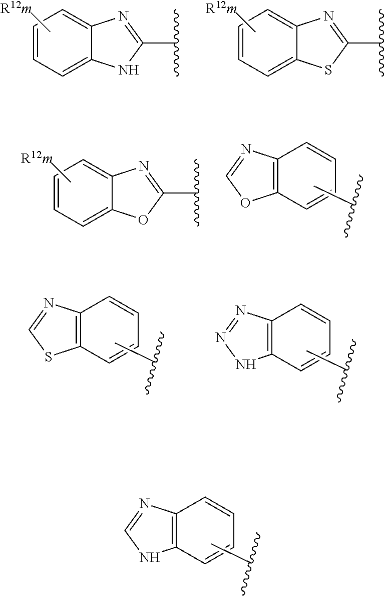
Benzofuranes 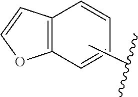
Quinolines isoquinolines, quinoxalines and quinazolines and related systems 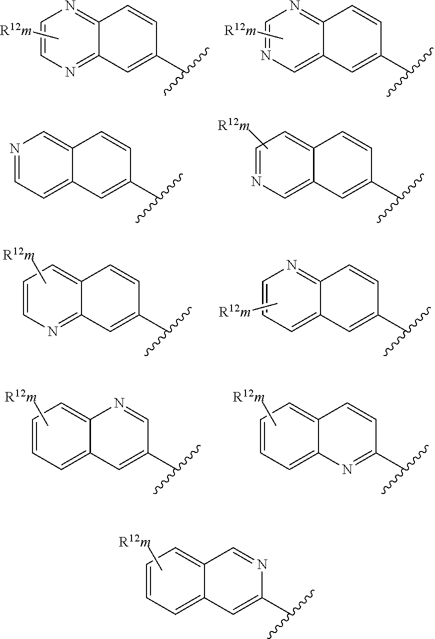
Qinolones 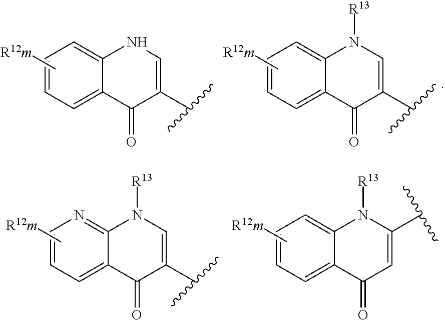
oxochromenes 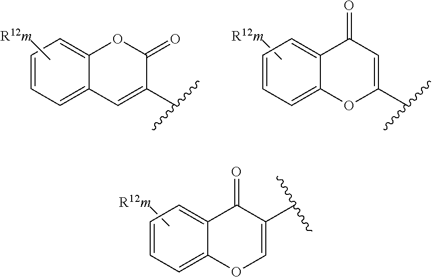
Tetrahydroisoquinoline 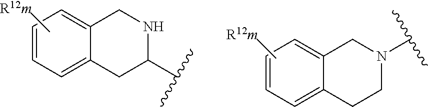
Wherein m of R¹² is any of 0-6, preferably 0, 1, 2, 3, 4, more preferably 0, 1, 2, 3;
Wherein R¹² is selected from —OH, —OC₁-C₆ alkyl, —C₁-C₆ alkyl, —F, —Cl, —NR₂, —(CH₂)ₐNR₂, —O(CH₂)ₙNR₂ with R being H or —C1-C6 alkyl and n being 1 or 2, in particular from OH, —CH₃, —C₂H₅, —OCH₃, —OC₂H₅, or two of R¹² form an acetal moiety,
Wherein R¹³ is selected from —C1-C6 alkyl.

3. The compound according to claim 1, wherein L₁ is a five membered aromatic N-heterocycle, in particular a substituted or unsubstituted imidazole or triazole, most preferably a triazole.

4. The compound according to claim 1, wherein n of R¹⁰ₙ and n of R¹¹ₙ being 0, 1, 2, 3 or 4, in particular n of R¹⁰ₙ and n of R¹¹ₙ being 0, 1, 2 or 3, and with each R¹⁰ and with each R¹¹ independently from any other R¹⁰ being selected from —OH, —OCH₃, —OC₂H₅ or -OiPr, particularly with one R¹⁰ or R¹¹ being —OH and the other R¹⁰ or R¹¹ being —OCH₃, —OC₂H₅ or -OiPr respectively.

5. The compound according to claim 1, having the general formulae (1a) 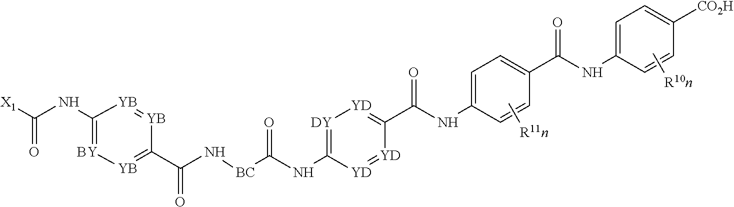
with YB being independently from each other CF, CCl or CH,
with YD being independently from each other N, CF, CCl or CH, in particular N and CH,
with X1, BC, R¹⁰ₙ and R¹¹ₙ as in one of the preceding claims.

6. The compound according to claim 1, having the general formulae (2) 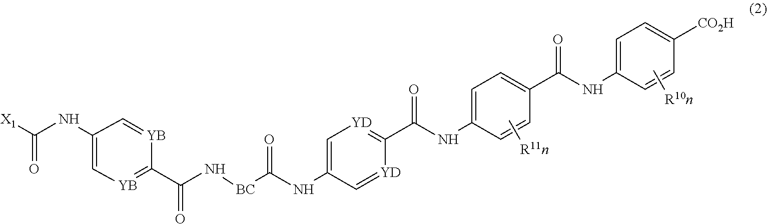
with YB being independently from each other CF, CCl or CH,
with YD being independently from each other N, CF, CCl or CH, in particular N and CH,
with X1, BC, R¹⁰ₙ and R¹¹ₙ as in one of the preceding claims.

7. The compound according to claim 1, having the general formulae (3) 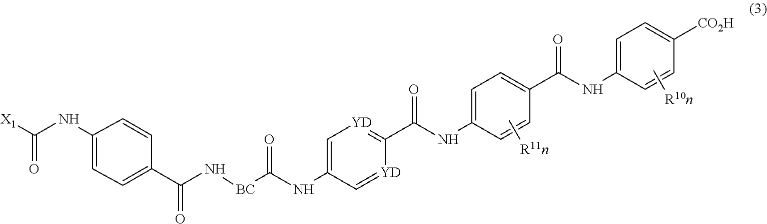
with YD being independently from each other N, CF, CCl or CH, in particular N and CH,
with X1, BC, R¹⁰ₙ and R¹¹ₙ as in one of the preceding claims.

8. The compound according to claim 1, having the general formulae (4) 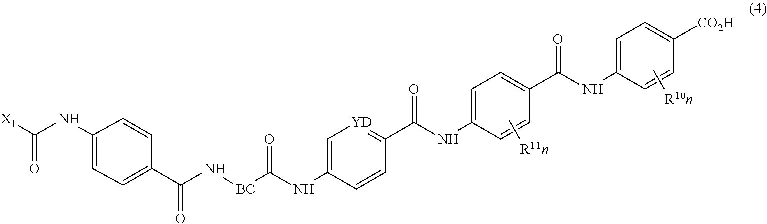
with YD being N,
with L1 being a tetrazole or imidazole, preferably tetrazole,
with X1, R¹⁰ₙ and R¹¹ₙ as in one of the preceding claims.

9. The compound according to claim 1, having the general formulae (5) 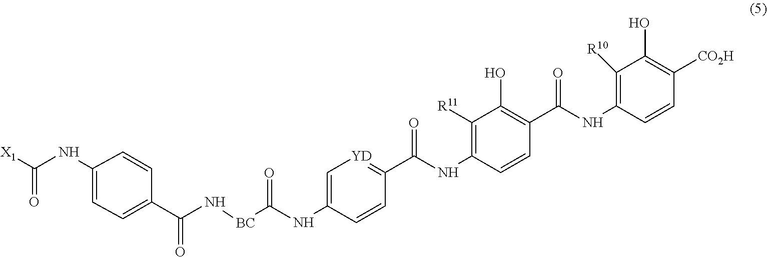
with YD being N,
with L₁ being a triazole or imidazole, preferably triazole,
with X1, R¹⁰ₙ and R¹¹ₙ as in one of the preceding claims.

10. The compound according to claim 5, wherein X1 is one of the following

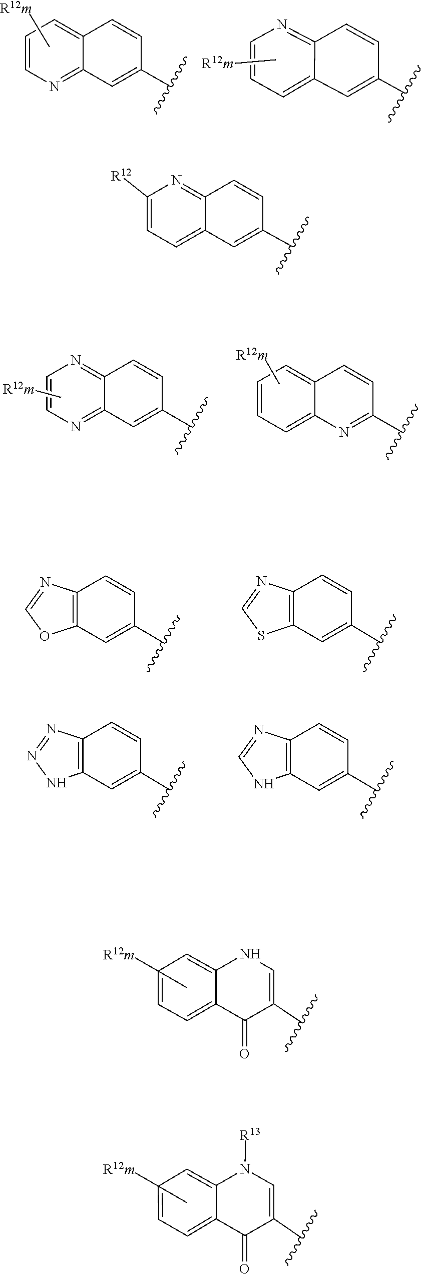

11. The compound according to claim 2, wherein
R¹² is selected from —OH, —CH₃, —C₂H₅, —OCH₃, —OC₂H₅, in particular —OH and —CH₃, or two of R¹² form —O—CH₂—O— moiety,

12. The compound according to claim 2 wherein R¹³ is selected from —CH₃, —C₂H₅, —C₃H₇.

13. The compound according to claim 1, wherein X1 is one of the following

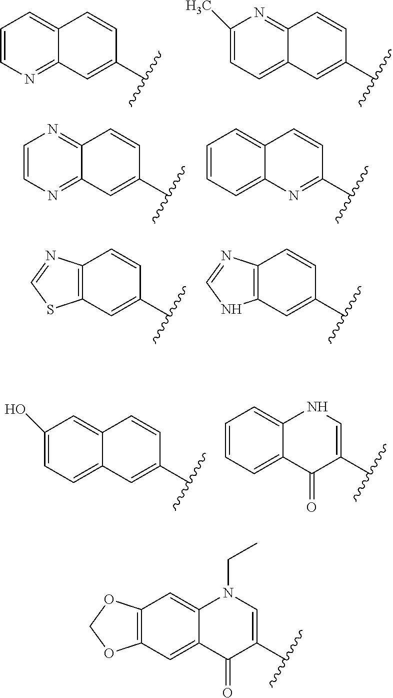

14. A method of treatment of diseases of a patient in need thereof by administering a compound according to claim 1 to said patient.

15. The method according to claim 15, wherein the bacterial infection is an infection by one of the genus Acinetobacter, Bordatella, Borellia, Brucella, Camphylobacter, Chlamydia, Chlamydophila, Enterobacter, Escherichia, Francisella, Haemophilus, Helicobacter, Klebisella, Legionella, Leptospira, Morganella Moraxella, Neisseria, Proteus, Pseudomonas, Rickettsia, Shigella, Salmonella, Stenotrophomonas, Treponema or Yersinia, Bacillus, Chlostridium, Corynebacterium, Enterococcus, Listeria, Micrococcus, Staphylococcus or Streptococcus Mycobacterium, Mycoplasmataceae, in particular Escherichia, Bacillus, Salmonella, Micrococcus, Mycobacterium.

16. The method according to claim 14, wherein said treatment is a treatment of bacterial infections by gram-negative or gram-positive bacterial strains.
